(19) United States
(12) Patent Application Publication
(10) Pub. No.: US 2024/0190993 A1
(43) Pub. Date: Jun. 13, 2024

(54) NEW METHOD TO IMPROVE THE ANTI-TUMORAL ACTIVITY OF MACROPHAGES

(71) Applicants: INSERM (INSTITUT NATIONAL DE LA SANTÉ ET DE LA RECHERCHE MÉDICALE), Paris (FR); OGD2 PHARMA, Nantes (FR); NANTES UNIVERSITÉ, Nantes (FR)

(72) Inventors: Stéphane BIRKLE, Nantes (FR); Sarah VERMEULEN, Nantes (FR); Sophie FOUGERAY, Nantes (FR); Meriem BAHRI, Nantes (FR); Emilie MADURA, Nantes (FR); Jean-Marc LE DOUSSAL, Nantes (FR)

(73) Assignees: INSERM (INSTITUT NATIONAL DE LA SANTÉ ET DE LA RECHERCHE MÉDICALE), Paris (FR); OGD2 PHARMA, Nantes (FR); NANTES UNIVERSITÉ, Nantes (FR)

(21) Appl. No.: 18/555,093

(22) PCT Filed: Apr. 13, 2022
(86) PCT No.: PCT/EP2022/059941

§ 371 Date: Oct. 12, 2023

(30) Foreign Application Priority Data
Apr. 14, 2021 (EP) 21305491.9

(51) Int. Cl. C07K 16/30; A61K 31/203; A61K 39/00; A61P 35/00; C07K 14/55; C07K 16/28

(52) U.S. Cl. CPC C07K 16/3084; A61K 31/203; A61P 35/00; C07K 14/55; C07K 16/2803; C07K 16/2896; A61K 2039/507; C07K 2317/24; C07K 2317/31; C07K 2317/565

(57) ABSTRACT

The treatment of cancer and particularly the neuroblastoma, and a combination of an anti-O-acetylated disialoganglioside (OAcGD2) compound and an anti-SIRP-alpha/CD47 compound for treating a cancer in a subject in need thereof. The inventors hypothesized that CD47 expression would interfere with the contribution of macrophage to the therapeutic effect of anti-OAcGD2 mAbs. They found that NB cells up-regulate CD47 expression upon 8B6 mAb immunotherapy in vivo, allowing them to escape mAb 8B6-mediated ADP. Next, they demonstrate that an anti-SIRPα antibody that blocks the binding of CD47 to SIRPα enables macrophages to phagocyte anti-OAcGD2-opsonised CD47-expressing NB cells in vitro. As a result, the combination of CD47 blocking with the targeting of OAcGD2-positive NB cells greatly reduces tumor growth in syngeneic mice. These results suggest that the combination of anti-OAcGD2 mAbs with phagocytosis checkpoints inhibitors may represent a very effective regimen to achieve for lasting responses in a greater number of patients.

### FIELD OF THE INVENTION

The present invention relates to a combination of an anti-O-acetylated disialoganglioside (OAcGD2) compound and an anti-SIRP-alpha/CD47 compound for use in the treatment of a cancer in a subject in need thereof.

### BACKGROUND OF THE INVENTION

Neuroblastoma (NB) is a cancer of the sympathetic nervous system derived from primordial neural crest cells that accounts for 15% of all childhood deaths (1). It is characterized by a highly heterogeneous clinical behavior, ranging from spontaneous regression to rapid progression and patient death (1). With an unfavorable prognosis, high-risk NB that occurs in half of all patients is associated with metastasis and with amplification of the MYCN oncogene, the major oncogenic driver (2, 3). Currently, the treatment for these patients consists in an aggressive multimodal therapy including surgery, radiation and high dose chemotherapy followed by stem cell rescue followed by maintenance therapy consisting in 13-cis-retinoic acid (13-cis-RA) in combination with interleukin (IL-2) and the anti-disialoganglioside GD2 monoclonal antibody (mAb) (4, 5). Although this regimen is now the standard of care, both neuropathic pain induction, associated with anti-GD2 therapeutic mAb infusion, and IL-2 related toxicities, remain a clinical challenge (4, 5). In addition, a relevant proportion of patients do not profit from this therapy (4, 5). The reasons for this treatment failure are not known. Consequently, strategies to further enhance the efficiency of this therapy are important to improve the patient care and the survival rate.

The anti-tumor effect of this therapy is mainly executed through antibody-dependent cell-mediated cytotoxicity (ADCC) initiated by the engagement of FcgammaRIIIa (CD16) on natural killer (NK) lymphocytes (6, 7). Major efforts are, therefore, been made to identify to strategy to enhance the efficiency of this mode of action to increase anti-NB responses (8). However, reports suggesting the implication of granulocytes and macrophages through the engagement of FcgammaRIIA receptor (CD32) exist (9, 10); the relative contribution of these effector cell populations to the therapeutic effect is not exactly clear (11).

Next to ADCC through the engagement of FcgammaRIIA receptor (CD32), macrophages may also phagocyte entire tumor cells (12). This beneficial activity is however frequently suppressed by the signal regulatory protein-alpha (SIRPα)-CD47 pathway, a phagocytosis checkpoint in macrophages and innate immune cells (13). In normal healthy cells, CD47 interacts with its receptor SIRPα, to trigger an anti-phagocytic “do not eat me” signal that prevents macrophages from engulfing target cells (14). Over-expression of CD47 also allows cancer cells to evade this innate immune surveillance mechanism (13), and blocking CD47-SIRPα interaction with an anti-CD47 blocking antibody enables macrophages to phagocytosis and eliminate cancer cells (15). Yet, CD47-SIRPα blockade alone is inefficient to enable tumor cell phagocytosis and require the concomitant engagement of a prophagocytic receptor such as Fc receptors (16). This can be achieved through the Fc region of either the CD47 blocking antibody (17). However, wide distribution of CD47 on normal cells may represent potential sites of toxicity and an “antigen sink” that could prevent CD47-targeting agents from reaching tumor cells at therapeutically relevant doses (18). With a more restricted expression pattern, SIRPα may be preferable from a safety standpoint (19).

The inventors focus on immunotherapeutic strategies targeting the O-acetylated form of GD2 (OAcGD2), which they believed could address the critical neuropathic pain side effect associated with anti-GD2 mAb infusions. They reported previously that mAb 8B6 specific for OAcGD2 displays antitumor activity in NB tumor models, with induction of ADCC similarly to anti-GD2 mAbs (20, 21, 22). Importantly, anti-OAcGD2 mAbs do not bind to peripheral nerves contrary to anti-GD2 therapeutic antibodies, and, by contrast to anti-GD2 mAbs, 8B6 mAb does not induce pain sensitization in rats (22).

### SUMMARY OF THE INVENTION

The inventors hypothesized that CD47 expression would interfere with the contribution of macrophage to the therapeutic effect of anti-OAcGD2 mAbs. They found that NB cells up-regulate CD47 expression upon 8B6 mAb immunotherapy in vivo, allowing them to escape mAb 8B6-mediated ADP. Next, they demonstrate that an anti-SIRPα antibody that blocks the binding of CD47 to SIRPα enables macrophages to phagocyte anti-OAcGD2-opsonised CD47-expressing NB cells in vitro. As a result, the combination of CD47 blocking with the targeting of OAcGD2-positive NB cells greatly reduces tumor growth in syngeneic mice. These results suggest that the combination of anti-OAcGD2 mAbs with phagocytosis checkpoints inhibitors may represent a very effective regimen to achieve for lasting responses in a greater number of patients.

Thus, the present invention relates to a combination of an anti-O-acetylated disialoganglioside (OAcGD2) compound and an anti-SIRP-alpha/CD47 compound for use in the treatment of a cancer in a subject in need thereof. Particularly, the invention is defined by its claims.

### DETAILED DESCRIPTION OF THE INVENTION

As used herein, the expression “anti-SIRP-alpha/CD47 compound” means “an anti-SIRP-alpha compound or an anti-CD47 compound”. Similarly, the expression “anti-SIRP-alpha/CD47 antibody” means “an anti-SIRP-alpha antibody or an anti-CD47 antibody”.

The present invention relates to a combination of an anti-O-acetylated disialoganglioside (OAcGD2) compound and an anti-SIRP-alpha/CD47 compound for use in the treatment of a cancer in a subject in need thereof.

In another embodiment, the invention relates to i) an anti-OAcGD2 and ii) anti-SIRP-alpha/CD47 compound, as a combined preparation for simultaneous, separate or sequential use in the treatment of cancer in a subject in need thereof.

In a particular embodiment, a retinoic acid can be added to the combination of an anti-O-acetylated disialoganglioside (OAcGD2) compound and an anti-SIRP-alpha/CD47 compound.

In a particular embodiment, the invention relates to a combination of an anti-O-acetylated disialoganglioside (OAcGD2) compound and an anti-SIRP-alpha/CD47 compound to improve the phagocytosis of macrophage cells.

In a particular embodiment, the invention relates to a combination of an anti-O-acetylated disialoganglioside (OAcGD2) compound and an anti-SIRP-alpha/CD47 compound to improve the activity of macrophage cells.

In a particular embodiment, the invention relates to i) an anti-OAcGD2 and ii) anti-SIRP-alpha/CD47 compound and iii) a retinoic acid, as a combined preparation for simultaneous, separate or sequential use in the treatment of cancer in a subject in need thereof.

In a particular embodiment, the invention relates to a combination of an anti-O-acetylated disialoganglioside (OAcGD2) compound and an anti-SIRP-alpha/CD47 compound to inhibit the escape of the cancerous cells to an anti-O-acetylated disialoganglioside therapy.

As used herein the term “O-acetylated disialoganglioside” (“OAcGD2”) refers to an O-acetylated form of the disialoganglioside GD2. Alternative names for OAcGD2 include “O-acetylated-GD2 ganglioside”, “O-acetyl GD2 ganglioside” and “O-acetyl GD2”, as non-limiting examples. As used herein, the expressions “OAcGD2”, “O-acetylated-GD2 ganglioside”, “O-acetyl GD2 ganglioside” and “O-acetyl GD2” are used indifferently. OAcGD2 is a molecule expressed in cancer tissues, including human neuroblastoma and melanoma, with highly restricted expression on normal tissues, principally to the cortex and peripheral nerves in humans.

As used herein, the term “retinoic acid” denotes a metabolite of vitamin A1 (all-trans-retinol) that mediates the functions of vitamin A1 required for growth and development.

According to the invention, the retinoic acid can be the 9-cis-retinoic acid (also known as alitretinoin) or the 13-cis-retinoic acid (also known as isotretinoin). Particularly, the retinoic acid is the 13-cis-retinoic acid.

According to the invention, the cancer may be selected in the group consisting of adrenal cortical cancer, anal cancer, bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, uterine cancer, cervix cancer, colon cancer, melanoma, neuroendocrine cancer, head and neck cancer, and soft tissue tumors.

In a particular embodiment, the cancer is a neuroblastoma, a glioblastoma, a small-cell lung carcinoma or a breast cancer. More particularly, the cancer is a neuroblastoma.

In a particular embodiment, the cancer is a cancer expressing OAcGD2. As used herein, the term “cancer expressing OAcGD2” refers to cancer having cells expressing the O-acetylated form of GD2 ganglioside on their surface. Typically, said cells express more than 1,000 OAcGD2 ganglioside molecules on their cell surface, preferably more than 10,000, and more preferably more than 50,000 OAcGD2 ganglioside molecules on their cell surface. Said cancer expressing the OAcGD2 ganglioside are selected from the group comprising or consisting of neuroblastoma, glioma, retinoblastoma, Ewing's family of tumors, sarcoma (i.e. rhabdomyosarcoma, osteosarcoma, leiomyosarcoma, liposarcoma, and fibrosarcoma), small cell lung cancer, breast cancer, melanoma, metastatic renal carcinoma, head and neck cancer and hematological cancers (i.e. leukemia, Hodgkin lymphoma, non-Hodgkin lymphoma and myeloma). More generally, term “cancer expressing the OAcGD2 ganglioside” refers to cancer presenting more than 10% of cells expressing the OAcGD2 ganglioside, preferably more than 15%, and still more preferably more than 20%. Preferably, said cells are Cancer Stem Cells (CSCs). “Treatment” of cancer expressing the OAcGD2 ganglioside refers to the administration of the composition comprising: (i) at least one anti-cancer agent, (ii) at least one antibody recognizing the OAcGD2 ganglioside, a functional fragment or a derivative thereof, and optionally (iii) a pharmaceutically acceptable carrier to destroy the tumor or to destroy cancer cells expressing the OAcGD2 ganglioside on their surface or a retinoic acid.

As used herein, the term “subject” denotes a mammal, such as a rodent, a feline, a canine, and a primate. Particularly, the subject according to the invention is a human. More particularly, the human suffers of a cancer. In some embodiments, a subject may be a patient, who is awaiting the receipt of, or is receiving medical care or was/is/will be the object of a medical procedure, or is monitored for the development of a disease.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the terms “prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

According to the invention, the term “compounds of the invention” denotes the anti-O-acetylated disialoganglioside (OAcGD2) compound alone or combined to a retinoic Acid (RA) compound and the anti-SIRP-alpha/CD47 compound.

The terms “anti-O-acetylated disialoganglioside (OAcGD2) compound” denotes any compound (molecule) which target the OAcGD2 (gene or protein) and inhibits its function and/or its interaction with others molecules. According to the invention, the anti-OAcGD2 compound can be an antibody which directly targets the OAcGD2 and can also be a functional fragment or a derivative thereof of a specific antibody. Alternatively, the anti-OAcGD2 compound may be a nucleic acid molecule encoding an antibody or an antigen-binding fragment that binds the OAcGD2. More particularly, the nucleic acid molecule may be a DNA or RNA, e.g. a mRNA, molecule encoding an antigen-binding fragment, such as a heavy chain variable region or a light chain variable region of an antibody, that binds the OAcGD2.

The term “an anti-SIRP-alpha/CD47 compound” denotes any compound (molecule) which will block the interaction between SIRP-alpha and CD47 or which will inhibit the activity or the expression of SIRP-alpha and/or CD47. According to the invention, the anti-SIRP-alpha/CD47 can be an antibody which directly target SIRP-alpha or CD47 and can also be a functional fragment or a derivative thereof of a specific antibody. Alternatively, the anti-SIRP-alpha/CD47 compound may be a nucleic acid molecule encoding an antibody or an antigen-binding fragment that binds SIRP-alpha or CD47. More particularly, the nucleic acid molecule may be a DNA or RNA, e.g. a mRNA, molecule encoding an antigen-binding fragment, such as a heavy chain variable region or a light chain variable region of an antibody, that binds SIRP-alpha or CD47.

According to the invention, anti-O-acetylated disialoganglioside (OAcGD2) compound (alone or combined with RA) and anti-SIRP-alpha/CD47 compound can be a multi-specific antibody, particularly a bi-specific antibody according to the invention or a derivative thereof.

As used herein, the term “SIRP-alpha” for “Signal Regulatory Protein alpha” denotes a regulatory membrane glycoprotein from SIRP family expressed mainly by myeloid cells and also by stem cells or neurons. SIRPα acts as inhibitory receptor and interacts with a broadly expressed transmembrane protein CD47 also called the “don't eat me” signal. This interaction negatively controls effector function of innate immune cells such as host cell phagocytosis. SIRP-alpha diffuses laterally on the macrophage membrane and accumulates at a phagocytic synapse to bind CD47 and signal ‘self’, which inhibits the cytoskeleton-intensive process of phagocytosis by the macrophage. This is analogous to the self signals provided by MHC class I molecules to NK cells via Ig-like or Ly49 receptors.

As used herein, the term “CD47” for “Cluster of Differentiation 47” also known as “Integrin Associated Protein” (IAP) denotes a transmembrane protein that in humans is encoded by the CD47 gene. CD47 belongs to the immunoglobulin superfamily and partners with membrane integrins and also binds the ligands thrombospondin-1 (TSP-1) and signal-regulatory protein alpha (SIRP-alpha). CD47 acts as a don't eat me signal to macrophages of the immune system which has made it a potential therapeutic target in some cancers, and more recently, for the treatment of pulmonary fibrosis.

In order to test the functionality of a putative anti-SIRP-alpha or CD47 compound a test is necessary. For that purpose, to identify anti-SIRP-alpha or CD47 compound, a flow cytometry test can be provided. After incubation with the compound, SIRP-alpha or CD47 cell surface expression will be studied by flow cytometry analysis. To this end, cells will be collected, stained with either CD47− or SIRP-alpha-specific monoclonal antibody. Cell-bound monoclonal antibody will be then detected using a fluorescent-labelled secondary antibody by flow cytometry analysis.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immuno-specifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs.

As used herein, an antibody or binding fragment thereof is said to be “immunospecific”, “specific for” or to “specifically bind” an antigen if it reacts at a detectable level with said antigen, preferably with an affinity constant (K_{A}) of greater than or equal to about 10⁵ M⁻¹, preferably greater than or equal to about 10⁶ M⁻¹, 10⁷ M⁻¹, 10⁸ M⁻¹, 5×10⁸ M⁻¹, 10⁹ M⁻¹, 5×10⁹ M⁻¹ or more.

Affinity of an antibody or binding fragment thereof for its cognate antigen is also commonly expressed as an equilibrium dissociation constant (K_{D}). an antibody or binding fragment thereof is said to be “immunospecific”, “specific for” or to “specifically bind” an antigen if it reacts at a detectable level with said antigen, preferably with a K_{D} of less than or equal to 10⁻⁵ M, preferably less than or equal to 10⁻⁶ M, 10⁻⁷ M, 5×10⁻⁸ M, 10⁻⁸ M, 5×10⁻⁹ M, 10⁻⁹ M or less.

Affinities of antibodies or binding fragment thereof can be readily determined using conventional techniques, for example, those described by Scatchard, 1949. Ann NY Acad Sci. 51:660-672. Binding properties of an antibody or binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®) or by BioLayer Interferometry (BLI).

As used herein, the term “antibody” refers to both intact immunoglobulin molecules as well as fragments thereof that include the antigen-binding site, and includes polyclonal, monoclonal, genetically engineered and otherwise modified forms of antibodies, including but not limited to chimeric antibodies, humanized antibodies, heteroconjugate, multispecific antibodies (e.g., bispecific, trispecific or tetraspecific antibodies, diabodies, tribodies, and tetrabodies) and polypeptide-Fc fusions.

The term “antibody” as used herein also refers to antibody fragment or to an antigen-binding fragment derived directly or indirectly from immunoglobulins, such as for example a portion of an antibody, such as F(ab′)2, F(ab)2, Fab′, Fab, Fv, single-chain Fvs (scFv), single-chain antibodies, disulfide-linked Fvs (sdFv), single-domain antibodies, Fd, defucosylated antibodies, fragments comprising either a VL or VH domain, fragments produced by a Fab expression library, and anti-idiotypic (anti-id) antibodies. The term “antibody fragment” includes DARTs, and diabodies, triabodies, tetrabodies, or any other synthetic or genetically engineered proteins comprising immunoglobulin variable regions that act like an antibody by binding to a specific antigen to form a complex. The term “single-chain antibodies” also includes single heavy chain variable domains of antibodies of the type that can be found in Camelid mammals commonly known as VHH.

Thus, the term “antibody” herein also refers to single chain variants including scFv fragments, VHHs, Trans-bodies®, Affibodies®, shark single domain antibodies, single chain or Tandem diabodies (TandAb®), Anticalins®, Nanobodies®, minibodies, BiTE®s, bicyclic peptides and other alternative immunoglobulin protein scaffolds.

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding.

“Fv”, as used herein, refers to the minimum antibody fragment that contains a complete antigen-recognition and -binding site. This fragment consists of a dimer of one HCVR and one LCVR in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Diabodies”, as used herein, refers to small antibody fragments prepared by constructing scFv fragments with short linkers (about 5-10 residues) between the HCVR and LCVR such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the HCVR and LCVR of the two antibodies are present on different polypeptide chains.

Antibody binding fragments can be obtained using standard methods. For instance, Fab or F(ab′)2 fragments may be produced by protease digestion of the isolated antibodies, according to conventional techniques.

It will also be appreciated that antibodies or binding fragments thereof according to the present invention can be modified using known methods. For example, to slow clearance in vivo and obtain a more desirable pharmacokinetic profile, the antibody or binding fragment thereof may be modified with polyethylene glycol (PEG).

“Unibodies” are well-known in the art and refer to antibody fragments lacking the hinge region of IgG4 antibodies. The deletion of the hinge region results in a molecule that is essentially half the size of traditional IgG4 antibodies and has a univalent binding region rather than the bivalent biding region of IgG4 antibodies.

“Domain antibodies” are well-known in the art and refer to the smallest functional binding units of antibodies, corresponding to the variable regions of either the heavy or light chains of antibodies.

“Single-domain antibodies” are well-known in the art and refer to antibody-derived proteins that contain the unique structural and functional properties of naturally-occurring heavy chain antibodies. These heavy chain antibodies may contain a single variable domain (VHH)—one such example is Nanobodies®—, or a single variable domain (VHH) and two constant domains (C_{H}2 and C_{H}3)—such as camelid antibodies—, or a single variable domain (VHH) and five constant domains (C_{H}1, C_{H}2, C_{H}3, C_{H}4 and C_{H}5)—such as shark antibodies.

In one embodiment, the antibody or binding fragment thereof according to the present invention also encompasses multispecific antibodies or binding fragments thereof, i.e., being immunospecific for more than one, such as at least two, different antigens.

Antibodies anti-OAcGD2, anti-SIRP-alpha or anti-CD47 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against OAcGD2, SIRP-alpha or CD47 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-OAcGD2, anti-SIRP-alpha or anti-CD47 single chain antibodies. Compounds useful in practicing the present invention also include anti-OAcGD2, anti-SIRP-alpha or anti-CD47 antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to OAcGD2, SIRP-alpha or CD47.

Humanized anti-OAcGD2, anti-SIRP-alpha or anti-CD47 antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of OAcGD2, SIRP-alpha or CD47 are selected.

In a particular embodiment, the anti-OAcGD2 antibody according to the invention may be an antibody as explained in the patent application WO2008043777, WO2014177271, WO2015067375 or WO2018103884.

In a particular embodiment, the anti-OAcGD2 antibody is the mouse antibody 8B6 comprising:
(a) a heavy chain wherein the variable domain comprises a H-CDR1 having a sequence set forth as SEQ ID NO:1; a H-CDR2 having a sequence set forth as SEQ ID NO:2; a H-CDR3 having a sequence set forth as SEQ ID NO:3; and
(b) a light chain wherein the variable domain comprises a L-CDR1 having a sequence set forth as SEQ ID NO:4; a L-CDR2 having a sequence set forth as SEQ ID NO:5; a L-CDR3 having a sequence set forth as SEQ ID NO:6.

Complementarity Determining Regions (CDR) sequences of 8B6 antibody are indicated in the following table 1:

TABLE 1
(IMGT nomenclature, which is well known in the
art (The International Immunogenetics
Information System ®, LEFRANC et al, Nucleic
Acids Research, vol. 27, p: 209-212, 1999)).

| Name | SEQ ID NO: | SEQUENCE |
|---|---|---|
| 8B6 VH-CDR1 | 1 | EFTFTDYY |
| 8B6 VH-CDR2 | 2 | IRNRANGYTT |
| 8B6 VH-CDR3 | 3 | ARVSNWAFDY |
| 8B6 VL-CDR1 | 4 | QSLLKNNGNTFL |
| 8B6 VL-CDR2 | 5 | KVS |
| 8B6 VL-CDR3 | 6 | SQSTHIPYT |

Preferably, the anti-OAcGD2 antibody is a chimeric antibody, more preferably a humanized antibody or a human antibody.

In a particular embodiment, the anti-OAcGD2 antibody is a humanized antibody.

In some embodiments, the anti-OAcGD2 antibody may be a humanized antibody derived from the mouse antibody 8B6.

In a particular embodiment, the humanized antibody of the invention has the CDRs of the 8B6 antibody.

Thus, in some embodiments, the anti-OAcGD2 antibody has a sequence comprising:
heavy chain HC-CDR1 of sequence EFTFTDYY (SEQ ID NO: 1), HC-CDR2 of sequence IRNRANGYTT (SEQ ID NO: 2), HC-CDR3 of sequence ARVSNWAFDY (SEQ ID NO: 3), and
light chain LC-CDR1 of sequence QSLLKNNGNTFL (SEQ ID NO: 4), LC-CDR2 of sequence KVS (SEQ ID NO: 5), LC-CDR3 of sequence SQSTHIPYT (SEQ ID NO: 6).

In some embodiments, the anti-OAcGD2 antibody is a humanized antibody having a sequence comprising:
a light chain variable region (VL) of sequence SEQ ID NO: 7, or a sequence having a percentage of identity of at least 85% with SEQ ID NO: 7; and/or
a heavy chain variable region (VH) of sequence SEQ ID NO: 8, or a sequence having a percentage of identity of at least 85% with SEQ ID NO: 8.

Non-limiting examples of humanized anti-OAcGD2 antibody for instance include humanized antibodies having a sequence comprising:
a heavy chain variable region (VH) sequence selected from the group consisting of SEQ ID NO: 9 (“VH49A”), SEQ ID NO: 10 (“VH72A”), SEQ ID NO: 11 (“VH49BHS”), SEQ ID NO: 12 (“VH72BHNPS”), SEQ ID NO: 16 (“VH49BHSs”), SEQ ID NO: 17 (“VH72BHNPSs”), or a variant thereof; and/or
a light chain variable region (VL) sequence selected from the group consisting of SEQ ID NO: 13 (“VL30A”), SEQ ID NO: 14 (“VL28A”), SEQ ID NO: 15 (“VL28Bs01/A2”), SEQ ID NO: 18 (“VL30As”), SEQ ID NO: 19 (“VL28B01/A2”), or a variant thereof.

An illustrative and non-limiting example of a humanized anti-OAcGD2 antibody is for instance the humanized antibody having a sequence comprising:
a heavy chain (HC) sequence of SEQ ID NO: 32, or a variant thereof; and
a light chain (LC) sequence of SEQ ID NO:33, or a variant thereof.

A “variant” or “derivative” protein is defined as having a sequence identical to at least 80%, preferably at least 85%, more preferably at least 90%, even at least 95%, 96%, 97%, 98% or 99% of the reference sequence.

The amino acid residues of the antibody of the invention could be numbered according to the IMGT numbering system. The IMGT unique numbering has been defined to compare the variable domains whatever the antigen receptor, the chain type, or the species (Lefranc M.-P., “Unique database numbering system for immunogenetic analysis” Immunology Today, 18, 509 (1997); Lefranc M.-P., “The IMGT unique numbering for Immunoglobulins, T cell receptors and Ig-like domains” The Immunologist, 7, 132-136 (1999); Lefranc, M.-P., Pommié, C., Ruiz, M., Giudicelli, V., Foulquier, E., Truong, L., Thouvenin-Contet, V. and Lefranc, G., “IMGT unique numbering for immunoglobulin and T cell receptor variable domains and Ig superfamily V-like domains” Dev. Comp. Immunol., 27, 55-77 (2003)). In the IMGT unique numbering, the conserved amino acids always have the same position, for instance cysteine 23, tryptophan 41, hydrophobic amino acid 89, cysteine 104, phenylalanine or tryptophan 118. The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1, 111.1, 112.2, 111.2, 112.3, 111.3, etc. (http://www.imgt.org/IMGTScientificChart/Nomenclature/IMGT-FRCDRdefinition.html). As used herein, the term “amino acid sequence” has its general meaning and is a sequence of amino acids that confers to a protein its primary structure. According to the invention, the amino acid sequence may be modified with one, two or three conservative amino acid substitutions, without appreciable loss of interactive binding capacity. By “conservative amino acid substitution”, it is meant that an amino acid can be replaced with another amino acid having a similar side chain. Families of amino acid having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., glycine, cysteine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In some embodiments, the anti-SIRP-alpha compound according to the invention is an anti-SIRP-alpha antibody. Said anti-SIRP-alpha antibody may be for example an antibody as explained in the patent application WO2015138600. Anti-SIRP-alpha antibody may be the antibody ab53721 or ab116254 sold by Abcam or the KWAR23 antibody (see for example Ring N G et al, 2017 PNAS).

In some embodiments, the anti-SIRP-alpha antibody is selected from the group comprising, but not limited to, ab53721, ab116254, KWAR23, FSI-189 (GS-0189), ES-004, BI765063 (OSE-172), ADU1805, IBI397 (AL008) and CC-95251, or an antigen-binding fragment thereof.

In some embodiments, the anti-CD47 compound according to the invention is an anti-CD47 antibody. Said anti-CD47 antibody according to the invention may be for example an antibody as explained in the patent application WO2005044857, WO2017053423 or WO2017049251. Anti-CD47 antibody may be Hu5F9-G4 antibody for example.

In some embodiments, the anti-CD47 antibody is selected from the group comprising, but not limited to, Magrolimab (Hu5F9-G4, 5F9, GS-4721), Gentulizumab, AO-176, AK117, CC-90002, STI-6643, IBI188, or an antigen-binding fragment thereof.

In a preferred embodiment, the anti-CD47 is Magrolimab, or an antigen-binding fragment thereof.

In some embodiments, the anti-CD47 antibody for instance has a sequence comprising a VHH-CDR1 of sequence GIIFKIND (SEQ ID NO: 35), a VHH-CDR2 of sequence ASTGGDEA (SEQ ID NO: 36), a VHH-CDR3 of sequence TA VISTDRDGTEWRR (SEQ ID NO: 37).

An illustrative and non-limiting example of an anti-CD47 antibody is an antibody having a sequence comprising a VHH of sequence SEQ ID NO: 38, or a variant thereof.

In a particular embodiment, the antibodies of the invention (anti-SIRP-alpha, anti-CD47 or anti-OAcGD2 antibodies) can also be multi-specific antibodies with at least two antigen binding sites directed to OAcGD2, SIRP-alpha or CD47. Particularly, the multi-specific antibody may be a bi-specific antibody directed to OAcGD2 and SIRP-alpha or CD47.

As used herein, a “multispecific” binding protein or antibody is a binding protein that binds two or more antigens, and/or two or more different epitopes. A multispecific antibody that binds two antigens, and/or two different epitopes, is also referred to herein as a “bispecific” antibody. A multispecific antibody that binds three antigens, and/or three different epitopes, is also referred to herein as a “trispecific” antibody.

As used herein, the term “specificity” refers to the number of binding specificities of a binding protein, an epitope, an antigen-binding protein or an antibody. For example, the term “monospecific antibody” refers to an antibody that specifically binds to one antigen target. The term “bispecific antibody” refers to an antibody that specifically binds to two different antigen targets. The term “trispecific antibody” refers to an antibody that specifically binds to three different antigen targets. The term “tetraspecific antibody” refers to an antibody that specifically binds to four different antigen targets and so forth.

As used herein, the term “valency” refers to the number of binding sites of a binding protein, an epitope, an antigen-binding protein or an antibody. For example, the term “monovalent antibody” refers to an antibody that has one antigen-binding site. The term “bivalent antibody” or “divalent antibody” refers to an antibody that has two binding sites. The term “trivalent antibody” refers to an antibody that has three binding sites. The term “tetravalent antibody” refers to an antibody that has four binding sites. In particular embodiments, the bivalent antibody can bind to one antigen target. In other embodiments, the bivalent antibody can bind to two different antigen targets. In particular embodiments, the trivalent antibody can bind to one antigen target, i.e., is monospecific. In other embodiments, the trivalent antibody can bind to two different antigen targets, i.e., is bispecific. In other embodiments, the trivalent antibody can bind to three different antigen targets, i.e., is trispecific. In particular embodiments, the tetravalent antibody can bind to one antigen target, i.e., is monospecific. In other embodiments, the tetravalent antibody can bind to two different antigen targets, i.e., is bispecific. In other embodiments, the tetravalent antibody can bind to three different antigen targets, i.e., is trispecific. In other embodiments, the tetravalent antibody can bind to four different antigen targets, i.e., is tetraspecific.

In some embodiments, the antibody is a bispecific antibody or antibody fragment. In some embodiments, the antibody is a trispecific antibody or antibody fragment. In some embodiments, the antibody is a trispecific antibody.

In some embodiments, the multispecific antibody of the invention is a trivalent antibody comprising three antigen binding sites and collectively targeting OAcGD2 and SIRP-alpha or CD47.

In multispecific antibodies, multivalence targeting the same antigen, such as OAcGD2 antigen, may be necessary to conserve high binding capacity. Thus, preferably, the multispecific antibody of the invention is an at least trivalent antibody comprising at least two antigen binding sites targeting OAcGD2 and at least one antigen binding site targeting SIRP-alpha or CD47.

As used herein, the terms “bispecific antibody”, “bispecific Ab”, “BAb”, or the like, refer to an antibody that comprises one or two antigen(s) binding site (Fab) directed against a first antigen and one or two further binding site(s) directed against a second antigen.

The “antigen-binding sites” comprised in the multispecific antibody of the invention may any natural or engineered antigen-binding molecule such as, e.g.:
Fragment variable (Fv), including single chain fragment variable (scFv), tandem scFv, Diabodies, DART®, TandAbs, and bispecific Fv fusion antibodies with an Fc domain;
Fragment antigen binding (Fab);
Single-domain antibodies, including Nanobodies®, VHH, V-NAR and domain antibodies (dAbs).

In some embodiments, the term “antigen-binding site” corresponds to the arms of the Y-shaped structure, which consist each of the complete light chain paired with the VH and CH1 domains of the heavy chain, and are called the Fab fragments (for Fragment antigen binding).

Thus, a further aspect of the invention refers to a multispecific antibody comprising at least one first antigen-binding site that binds OAcGD2 and at least one second antigen binding site that binds SIRP-alpha or CD47.

In a particular embodiment, the multispecific antibody comprises a first antigen-binding site from an anti-OAcGD2 monoclonal antibody and at least one second antigen binding site from an anti-SIRP-alpha or anti-CD47 monoclonal antibody.

In some embodiments, the multispecific antibody of the invention is a bispecific antibody.

In some embodiments, the first antigen specifically bound by the multispecific antibody is OAcGD2. In some embodiments, the second antigen specifically bound by the multispecific antibody is SIRP-alpha. In some embodiments, the second antigen specifically bound by the multispecific antibody is CD47.

Thus, the present invention refers to a bispecific antibody comprising a first antigen-binding site that binds OAcGD2 and a second antigen-binding site that binds SIRP-alpha, in particular comprising a first Fab from an anti-OAcGD2 antibody and a second Fab from an anti-SIRP-alpha antibody.

Also, the present invention refers to a bispecific antibody comprising a first antigen-binding site that binds OAcGD2 and a second antigen-binding site that binds CD47, in particular comprising a first Fab from an anti-OAcGD2 antibody and a second Fab from an anti-CD47 antibody.

In some embodiments, the antigen-binding site that binds OAcGD2 comprises the CDR or the VH/VL sequences, or fragments, variants or derivatives thereof, of the anti-OAcGD2 antibodies as described hereinabove in the present specification.

Thus, in some embodiments, the antigen-binding site that binds OAcGD2 has a sequence comprising:
heavy chain HC-CDR1 of sequence EFTFTDYY (SEQ ID NO: 1), HC-CDR2 of sequence IRNRANGYTT (SEQ ID NO: 2), HC-CDR3 of sequence ARVSNWAFDY (SEQ ID NO: 3), and
light chain LC-CDR1 of sequence QSLLKNNGNTFL (SEQ ID NO: 4), LC-CDR2 of sequence KVS (SEQ ID NO: 5), LC-CDR3 of sequence SQSTHIPYT (SEQ ID NO: 6).

In some embodiments, the antigen-binding site that binds OAcGD2 has a sequence comprising:
a light chain variable region (VL) of sequence SEQ ID NO: 7, or a sequence having a percentage of identity of at least 85% with SEQ ID NO: 7; and/or
a heavy chain variable region (VH) of sequence SEQ ID NO: 8, or a sequence having a percentage of identity of at least 85% with SEQ ID NO: 8.

Non-limiting examples of antigen-binding sites that bind OAcGD2 for instance have sequences comprising:
a heavy chain variable region (VH) sequence selected from the group consisting of SEQ ID NO: 9 (“VH49A”), SEQ ID NO: 10 (“VH72A”), SEQ ID NO: 11 (“VH49BHS”), SEQ ID NO: 12 (“VH72BHNPS”), SEQ ID NO: 16 (“VH49BHSs”), SEQ ID NO: 17 (“VH72BHNPSs”), or a variant thereof; and/or
a light chain variable region (VL) sequence selected from the group consisting of SEQ ID NO: 13 (“VL30A”), SEQ ID NO: 14 (“VL28A”), SEQ ID NO: 15 (“VL28Bs01/A2”), SEQ ID NO: 18 (“VL30As”), SEQ ID NO: 19 (“VL28B01/A2”), or a variant thereof.

In some embodiments, the antigen-binding site that binds SIRP-alpha or CD47 comprises the CDR or the VH/VL sequences, or fragments, variants or derivatives thereof, of the anti-SIRP-alpha or anti-CD47 antibodies as described hereinabove in the present specification.

In some embodiments, the antigen-binding site that binds SIRP-alpha is an antigen-binding fragment of an anti-SIRP-alpha antibody selected from the group comprising, but not limited to, ab53721, ab116254, KWAR23, FSI-189 (GS-0189), ES-004, BI765063 (OSE-172), ADU1805, IBI397 (AL008) and CC-95251, or a derivative thereof.

In some embodiments, the antigen-binding site that binds CD47 is an antigen-binding fragment of an anti-CD47 antibody selected from the group comprising, but not limited to, Magrolimab (Hu5F9-G4, 5F9, GS-4721), Gentulizumab, AO-176, AK117, CC-90002, STI-6643, IBI188, or a derivative thereof.

In some embodiments, the antigen-binding site that binds CD47 has a sequence comprising a VHH-CDR1 of sequence GIIFKIND (SEQ ID NO: 35), a VHH-CDR2 of sequence ASTGGDEA (SEQ ID NO: 36), a VHH-CDR3 of sequence TAVISTDRDGTEWRR (SEQ ID NO: 37).

An illustrative and non-limiting example of an antigen-binding site that binds CD47 is an antigen-binding site having a sequence comprising a VHH of sequence SEQ ID NO: 38, or a variant thereof.

In a particular embodiment, a retinoic acid can be added to the multispecific antibody comprising a first antigen-binding site from an anti-OAcGD2 monoclonal antibody and at least one second antigen binding site from an anti-SIRP-alpha or anti-CD47 monoclonal antibody.

Exemplary formats for the multispecific antibody molecules of the invention include, but are not limited to (i) two antibodies cross-linked by chemical heteroconjugation, one with a specificity to OAcGD2 and another with a specificity to a second antigen, e.g SIRP-alpha or CD47; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary C_{H}3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the multispecific antibody comprises one or two Fc regions fused to one, two, three, or more antigen binding domains or other polypeptides (e.g, an Fc fusion protein).

Engineered antibodies with three or more antigen binding sites also include for example, “Octopus antibodies”, DVD-Ig, or “Dual Acting FAb”.

In some embodiments, the multispecific antibody is a dual variable domain (DVD) immunoglobulin, e.g., as described in WO2012061558. In some embodiments, the multispecific antibody comprises dual variable domains having a cross over orientation, e.g., as described in WO2012135345. In some embodiments, the multispecific antibody has a Y-shaped IgG like form, such as e.g. the “IgG configuration”, the “TBTI (tetravalent bispecific tandem immunoglobulin) configuration” or the “CODV (cross-over dual variable) configuration”).

Multispecific antibodies may also be provided in an asymmetric form with a domain crossover in one or more binding arms of the same antigen specificity, i.e. by exchanging the VH/VL domains or the complete Fab arms. In one aspect, the multispecific antibody comprises a cross-Fab fragment. The term “cross-Fab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing.

In some embodiments, the multispecific antibody comprises tandem Fabs. The term “tandem Fabs” refers to an antigen-binding protein, wherein the C terminus of one CHI region of a first Fab domain is operatively linked to the N terminus of a VH region of a second Fab domain. In certain embodiments, the tandem fab antibody may be tetravalent and monospecific (each of the four Fabs binding the same antigen). In certain embodiments, the tandem fab antibody may be tetravalent and bispecific (two of the four Fabs bind a first antigen or epitope while the other two fabs bind a second antigen or epitope).

The tandem Fabs may be operatively linked with any known peptide linker to the art used for linking two or more antigen-bind domains. In a particular embodiment, the peptide linker is a Gly-Ser linker, i.e., a linker comprising only glycine amino acid(s) and serine amino acid(s). Alternatively, or in combination with the above recited Gly-Ser linker, the peptide linker may comprise all or part of the sequence of the hinge region of one or more immunoglobulins selected from IgA, IgG, and IgD.

In some embodiments, the multispecific antibody comprises at least one pseudoFab moiety. As used herein, a “pseudoFab” moiety is analogous to a Fab moiety of a conventional antibody in that it comprises a functional antigen binding portion formed by the pairing of a variable light chain (VL) domain with a variable heavy chain (VH). However, whereas the VL and VH domains of a conventional Fab are directly fused with or linked to a constant light chain (CL) domain and a constant heavy chain 1 (CH1) domain, respectively, a pseudoFab moiety lacks CH1 and CL domains. Instead, the VL and VH domains of the pseudoFab are operatively linked to a second pair of stabilized knockout VL and VH domains (denoted herein as VLX and VHX) which form an inactive or non-functional binding portion (herein, a “stabilized knockout” portion or domain) that it is incapable of specifically binding to a target antigen (e.g., any target antigen). In certain embodiments, the pseudoFab moiety is incapable of binding the target antigen of the corresponding Fab moiety from which it is derived. The pseudoFab moiety lacks CH and CL domains.

While unable to selectively bind a target antigen, the VLX and VHX domains of a pseudoFab nevertheless preferentially associate which each other to form a stable chain pairing. Therefore, by appending a pseudoFab to one or more additional binding domains of differing specificities, the inherent stability of the VLX/VHX chain pairing of a pseudoFab can drive heterodimerization of the chains of a desired multispecific binding molecule.

Accordingly, a pseudoFab of the present disclosure comprises or consists of:
a first polypeptide chain having a structure represented by the formula:

(I)
VHa-L1-VHX;
or
(II)
VHb-L2-VHX

and a second polypeptide chain having a structure represented by the formula:

(III)
VLa-L3-VLX;
or
(IV)
VLb-L4-VLX

wherein
   VHX associates with VLX to form a knockout domain,
   VH associates with VL to form a first functional antigen binding domain, and
   L1, L2, L3 and L4 are linkers, which may present or absent, identical or different.

Other examples of bispecific antibody formats include, but are not limited to, the so-called “BITE” (bispecific T cell engager) molecules wherein two scFv molecules are fused by a flexible linker; diabodies and derivatives thereof, such as tandem diabodies (“TandAb”); “DART” (dual affinity retargeting) molecules which are based on the diabody format but feature a C-terminal disulfide bridge for additional stabilization, and so-called triomabs, which are whole hybrid mouse/rat IgG molecules.

Multispecific antibodies can be generated by combination of polypeptides chains targeting different antigens. The 2+1 or 2+2 antigen-binding valencies leading to tri or tetravalent molecules can be obtained from a whole IgG combined to antigen-binding building blocks derived from immunoglobulin domain of native antibodies such as single-domain antibody (SDA or VHH), variable fragment (Fv), single-chain variable fragment (scFv), Fab fragment, and single chain antigen-binding fragment, or bispecific antibody conjugates combining two whole IgG molecules linked together via a linker on Fc domain. Multispecific antibodies can also be designed with antigen-binding building blocks without the Fc domains.

Illustrative and non-limiting examples of bispecific multivalent antibody formats comprising an Fc domain include:
DVD-IgG constructs where monospecific IgG are designed for bispecificity via appending to the VL and VH domains of an IgG with one specificity, at the amino or carboxy terminus of either the light or heavy chain, with additional VL and VH of an IgG of different specificity;
1 scFv fusion proteins where addition of a new antigen-binding moiety (scFv) to full length IgG results in fusion proteins with tetravalency (available as C-terminal scFv fusion or N-terminal scFv fusion) leading to scFv(H)-IgG, scFv(L)-IgG, IgG(H)-scFv or IgG(L)scFv constructs;
Fab(H)-IgG where Fab is fused to the heavy chain of an IgG;
Diabody-Fc fusions where the Fab fragment of an IgG is replaced by a bispecific diabody;
scFv₄-IgG constructs containing two polypeptides, (scFv)₁-CL and (scFv)₂-CH1-CH2-CH3 leading to BsAb via the natural dimerization mechanism between IgG heavy and light chains;
IgG-scFab constructs which are engineered by fusing a scFab domain to the C-termini of IgG heavy chains modified by knobs-into-holes (KIH) technique.
DNL-Fab3 constructs which are trivalent antibodies composed of three Fab fragments joined by the use of the specific interaction between the regulatory subunits of cyclic adenosine monophosphate (cAMP)-dependent protein kinase A (PKA) and the anchoring domains of A kinase anchoring proteins (AKAP);
Fab-scFv-Fc where a Fab and a scFv are fused, separately, to the N-terminus of the heterodimerizing Fc chains.

Multispecific antibodies may be prepared as full-length antibodies or antibody fragments. Techniques for making multispecific antibodies include, but are not limited to, recombinant co-expression of two immunoglobulin heavy chain-light chain pairs having different specificities and “knob-in hole” engineering (see, e.g., U.S. Pat. No. 5,731,168). Nonlimiting exemplary knob-in-hole substitutions include T366W (knob) and T366S/L368A/Y407V (hole). In some embodiments, the knob-in-hole substitutions are in IgG1 constant domains.

Multispecific antibodies may also be made by engineering electrostatic steering effects for making antibody Fc-heterodimeric molecules. As a nonlimiting example, in a bispecific antibody comprising two heavy chain variable regions and two light chain variable regions, a first heavy chain variable region may comprise a Q39E substitution (Kabat numbering) and a first light chain variable region may comprise a Q38K substitution (Kabat numbering); and a second heavy chain variable region may comprise a Q39K substitution (Kabat numbering) and a second light chain variable region may comprise a Q38E substitution (Kabat numbering). In some embodiments, the Q39E/Q38K and Q39K/Q38E substitutions reduce mispairing of the heavy and light chains of the bispecific antibody. Similarly, a first heavy chain constant region may comprise a S183K substitution (EU numbering) and a first light chain constant region may comprise a V133E substitution (EU numbering), and a second heavy chain constant region may comprise a S183E substitution (EU numbering) and a second light chain constant region may comprise a V133K substitution (EU numbering). In some embodiments, the S183K/V133E and S183E/V133K substitutions reduce mispairing of the heavy and light chains of the bispecific antibody.

In some embodiments, a bispecific antibody comprises Q39E/Q38K and Q39K/Q38E substitutions in the binding domains and S183K/V133E and S183E/V133K substitutions in the constant regions. In some embodiments, a bispecific antibody comprises both knob-in-hole substitutions and electrostatic substitutions.

Multi-specific antibodies may also be made by cross-linking two or more antibodies or fragments; using leucine zippers to produce bi-specific antibodies (see WO 2011/034605); using the common light chain technology for circumventing the light chain mis-pairing problem (see WO 98/50431); using “diabody” technology for making bispecific antibody fragments; and using single-chain Fv (sFv) dimers.

In some embodiments, the bispecific antibody is obtained or obtainable via a controlled Fab-arm exchange, typically using DuoBody technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half-molecule” exchange (swapping of a heavy chain and attached light chain) between two monospecific antibodies, both comprising IgG4-like CH3 regions, upon incubation under reducing conditions. The resulting product is a bispecific antibody having two Fab arms which may comprise different sequences. In another exemplary method, described in WO 2011131746, bispecific antibodies of the present invention are prepared by a method comprising the following steps, wherein at least one of the first and second antibodies is the antibody of the present invention: a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is the antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl)phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety. The following are exemplary embodiments of combinations of such assymetrical mutations, optionally wherein one or both Fc-regions are of the IgG1 isotype.

In some embodiments, the first Fc region has an amino acid substitution at a position selected from the group consisting of: 366, 368, 370, 399, 405, 407 and 409, and the second Fc region has an amino acid substitution at a position selected from the group consisting of: 366, 368, 370, 399, 405, 407 and 409, and wherein the first and second Fc regions are not substituted in the same positions.

In some embodiments, the first Fc region has an amino acid substitution at position 405, and said second Fc region has an amino acid substitution at a position selected from the group consisting of: 366, 368, 370, 399, 407 and 409, optionally 409.

In some embodiments, the first Fc region has an amino acid substitution at position 409, and said second Fc region has an amino acid substitution at a position selected from the group consisting of: 366, 368, 370, 399, 405, and 407, optionally 405 or 368.

In some embodiments, both the first and second Fc regions are of the IgG1 isotype, with the first Fc region having a Leu at position 405, and the second Fc region having an Arg at position 409.

In some embodiments, the bispecific antibody is obtained or obtainable via a methods that maintains natural Fab structures of both original mAbs as well as full human Fc, described in Golay et al, 2016 (Golay et al, Design and Validation of a Novel Generic Platform for the Production of Tetravalent IgG1-like Bispecific Antibodies. J Immunol. 2016) and WO2013005194.

In some embodiment, the bispecific antibodies of the present invention comprises Fab fragments having mutations at the interface of the CHI and CL domains, said mutations preventing heavy chain/light chain mispairing.

In some embodiment, the CHI domain of the Fab fragments has mutations selected from the group consisting in: substitution of the threonine residue at position 192 with a glutamic acid residue; substitution of the leucine residue at position 143 with a glutamine residue and substitution of the serine residue at position 188; substitution of the leucine residue at position 124 with an alanine residue and substitution of the leucine residue at position 143 with a glutamic acid residue; and substitution of the valine residue at position 190 with an alanine residue.

In some embodiment, the CL domain of the Fab fragments has mutations selected from the group consisting in: substitution asparagine residue at position 137 with a lysine residue and substitution of the serine residue at position 114 with an alanine residue; substitution of the valine residue at position 133 with a threonine residue and substitution of the serine residue at position 176 with an valine residue; substitution of the valine residue at position 133 with a tryptophane residue; and substitution of the leucine residue at position 135 with a tryptophane residue and substitution of the asparagine residue at position 137 with an alanine residue.

In some embodiment, the bispecific antibodies of the present invention comprises Fab fragments having mutations at the interface of the CHI and CL domains, said mutations preventing heavy chain/light chain mispairing and said Fab fragments being tandemly arranged in any order, the C-terminal end of the CHI domain of the first Fab fragment being linked to the N-terminal end of the VH domain of the following Fab fragment through a polypeptide linker. Generally, said polypeptide linker should have a length of at least 20, preferably at least 25, and still more preferably at least 30, and up to 80, preferably up to 60, and still more preferably up to 40 amino-acids.

Advantageously, said polypeptide linker comprises all or part of the sequence of the hinge region of one or more immunoglobulin(s) selected among IgA, IgG, and IgD.

As used herein, the term “hinge region” includes the region of a heavy chain molecule that joins the C_{H}1 domain to the C_{H}2 domain. This hinge region comprises approximately 25 residues and is flexible, thus allowing the two N-terminal antigen binding regions to move independently.

In some embodiment, the polypeptide linker has a length of at least 20 amino-acids.

In some embodiment, the bispecific antibody of the invention has an immunoglobulin-like structure.

Furthermore, a multispecific antibody according to the invention may also be a fusion protein comprising at least two different antibody fragments or antigen-binding fragment with a specificity to OAcGD2 and another with a specificity to a second antigen, e.g SRIP-alpha or CD47; fused to another polypeptide, for example an Fc domain.

Furthermore, a multispecific antibody according to the invention may consist of a fusion protein comprising at least one VHH molecule with specificity to OAcGD2 and at least another with a specificity to a second antigen, e.g SIRP-alpha or CD47; fused to each other, with or without a linker, and/or fused to a further polypeptide, for example an Fc domain.

The anti-OAcGD2 compound may also be one or more nucleic acid molecule(s) encoding a multispecific antibody that binds OAcGD2. More particularly, said nucleic acid molecules may be DNA or RNA, e.g. mRNA, molecule(s) encoding a multispecific antibody that binds OAcGD2.

Similarly, the anti-SIRP-alpha/CD47 compound may also be one or more nucleic acid molecule(s) encoding a multispecific antibody that binds SIRP-alpha or CD47. More particularly, said nucleic acid molecules may be DNA or RNA, e.g. mRNA, molecules encoding a multispecific antibody that binds SIRP-alpha or CD47.

The present invention also provides antibodies comprising functional variants of the VL region, VH region, or one or more CDRs of the antibodies or multi-specific antibody of the invention. A functional variant of a VL, VH, or CDR used in the context of a monoclonal antibody of the present invention still allows the antibody to retain at least a substantial proportion (at least about 50%, 60%, 70%, 80%, 85%, 90%, 95% or more) of the affinity/avidity and/or the specificity/selectivity of the parent antibody and in some cases such a monoclonal antibody of the present invention may be associated with greater affinity, selectivity and/or specificity than the parent Ab. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation. Such functional variants typically retain significant sequence identity to the parent Ab.

“Identity” or “identical”, when used herein in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, WI; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

The sequence of CDR variants may differ from the sequence of the CDR of the parent antibody sequences through mostly conservative substitutions; for instance at least about 35%, about 50% or more, about 60% or more, about 70% or more, about 75% or more, about 80% or more, about 85% or more, about 90% or more, (e.g., about 65-95%, such as about 92%, 93% or 94%) of the substitutions in the variant are conservative amino acid residue replacements. The sequences of CDR variants may differ from the sequence of the CDRs of the parent antibody sequences through mostly conservative substitutions; for instance at least 10, such as at least 9, 8, 7, 6, 5, 4, 3, 2 or 1 of the substitutions in the variant are conservative amino acid residue replacements. In the context of the present invention, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of the antibodies of the invention. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 70% of identity to the parent peptide. According to the present invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence. According to the present invention a first amino acid sequence having at least 90% of identity with a second amino acid sequence means that the first sequence has 90; 91; 92; 93; 94; 95; 96; 97; 98; 99; or 100% of identity with the second amino acid sequence.

In another embodiment, the antibody according to the invention is a single domain antibody against OAcGD2, SIRP-alpha or CD47. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the anti-SIRP-alpha/CD47 compound according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In one embodiment, the anti-SIRP-alpha/CD47 compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of SIRP-alpha or CD47 compound are selected.

In another embodiment, the anti-SIRP-alpha/CD47 compound according to the invention is an inhibitor of SIRP-alpha or CD47 gene expression.

Small inhibitory RNAs (siRNAs) can also function as inhibitors of SIRP-alpha or CD47 expression for use in the present invention. SIRP-alpha or CD47 gene expression can be reduced by contacting a subject or cell with a small double stranded RNA (dsRNA), or a vector or construct causing the production of a small double stranded RNA, such that SIRP-alpha or CD47 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of SIRP-alpha or CD47 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of SIRP-alpha or CD47 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of SIRP-alpha or CD47 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing SIRP-alpha or CD47. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

In another embodiment, the invention relates to a method for treating a cancer comprising administering to a subject in need thereof a therapeutically effective amount of an anti-O-acetylated disialoganglioside (OAcGD2) compound and an anti-SIRP-alpha/CD47 compound.

In another embodiment, the invention relates to the use of combination of retinoic acid, an anti-O-acetylated disialoganglioside (OAcGD2) compound and an anti-SIRP-alpha/CD47 compound for the manufacturing of a medicament for the treatment of a cancer in a subject in need thereof.

In another embodiment, the invention relates to a pharmaceutical composition for the treatment of a cancer in a subject in need thereof, said pharmaceutical composition comprising retinoic acid, an anti-O-acetylated disialoganglioside (OAcGD2) compound and an anti-SIRP-alpha/CD47 compound.

According to the invention, the compounds of the invention can also be used in combination with at least one other therapeutic active agent as described below.

### Nucleic Acids, Vectors, Recombinant Host Cells and Uses Thereof

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the antibodies and polypeptides of the invention, it would be necessary to employ vectors comprising polynucleotide molecules for encoding the antibodies and polypeptides of the invention. Methods of preparing such vectors as well as producing host cells transformed with such vectors are well known to those skilled in the art.

Accordingly, a further object of the invention relates to a nucleic acid molecule encoding an antibody according to the invention. More particularly the nucleic acid molecule encodes a heavy chain or a light chain of an antibody of the present invention.

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. So, a further aspect of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like. Any expression vector for animal cell can be used, so long as a gene encoding the human antibody C region can be inserted and expressed. Examples of suitable vectors include PAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The choice of a suitable expression vector for expression of the antibodies of the invention will of course depend upon the specific host cell to be used, and is within the skill of the ordinary artisan. Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (e.g., a monoclonal antibody). Thus, a promoter nucleotide sequence is operably linked to a given DNA sequence if the promoter nucleotide sequence directs the transcription of the sequence.

A further aspect of the invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the present invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

### Therapeutic or Pharmaceutical Composition

Another object of the invention relates to a therapeutic or pharmaceutical composition comprising an anti-O-acetylated disialoganglioside (OAcGD2) compound and an anti-SIRP-alpha/CD47 compound according to the invention for use in the treatment of cancer in a subject in need thereof.

In some embodiment, the invention relates to a therapeutic or pharmaceutical composition comprising retinoic acid and a multi-specific antibody according to the invention for use in the treatment of cancer in a subject in need thereof.

In a particular embodiment, the invention relates to a therapeutic or pharmaceutical composition comprising retinoic acid and a bi-specific antibody comprising a first Fab from an anti-OAcGD2 antibody and a second Fab from an anti-SIRP antibody or comprising a first Fab from an anti-OAcGD2 antibody and a second Fab from an anti-CD47 antibody according to the invention for use in the treatment of cancer in a subject in need thereof.

In a particular embodiment, the invention relates to a therapeutic or pharmaceutical composition comprising retinoic acid and a fusion protein comprising at least two different antibody fragments or antigen-binding fragment with a specificity to OAcGD2 and another with a specificity to a second antigen, e.g SIRP-alpha or CD47; fused to another polypeptide, for example an Fc domain.

In a particular embodiment, the invention relates to a therapeutic or pharmaceutical composition comprising retinoic acid and a fusion protein comprising at least one VHH molecules with specificity to OAcGD2 and at least another with a specificity to a second antigen, e.g SIRP-alpha or CD47; fused to each other, with or without a linker, and/or fused to a further polypeptide, for example an Fc domain.

In a particular embodiment, a retinoic acid can be added to the therapeutic or pharmaceutical composition.

Any therapeutic agent of the invention may be combined with pharmaceutically acceptable excipients, and optionally sustained-release matrices, such as biodegradable polymers, to form therapeutic or pharmaceutical compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Particularly, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise at least one further therapeutic active agent. The present invention also relates to a kit comprising the compounds of the invention and a further therapeutic active agent.

For example, anti-cancer agents may be added to the pharmaceutical composition as described below.

Anti-cancer agents may be agents already used for example in neuroblastoma like interline 2 (IL-2) and antibody anti-disialoganglioside (anti-GD2). This agent can also be used in combination with the compounds of the invention.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family.

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The invention will be further illustrated by the following figures and examples. However, these examples and figures should not be interpreted in any way as limiting the scope of the present invention.

### FIGURES

FIG. 1. Importance of macrophage for the anti-tumor effect of OAcGD2-specific 8B6 mAb in vivo. (A) Mice (8 animal/group) were inoculated i.v. with 2.5×10⁵ NXS2 cells on day 0. Antibody 8B6 treatment was given i.v. on days 3, 7, 10, 14, 17, and 21. 13-cis-RA (10 mg/kg) was given per os, daily on day 17 to 21. Mice were euthanized on day 27 days for treatment efficacy analysis. (B) Anti-tumor efficacy was evaluated by determining the liver weight on the fresh specimen. The y-axis starts at 0.8 g corresponding to the aver-age of normal liver weight. 13-cis-RA enhances the protective effect of mAb 8B6. (C-E) Liver metastasis isolated from mice in (A) were analyzed for macrophage infiltration, CD47 expression, and SIRPα expression. (C) Plots showing the gating strategy used for flow cytometric identification of CD45+F4/80+CD11b+Ly6G-Ly6Clow-int cells in liver metastasis. (D) Fold increase of total count of macrophages in liver metastasis were achieved by flow cytometry of CD45+F4/80+CD11b+Ly6G-Ly6Clow-int gated cells, as indicated. Mice treated with either mAb 8B6 alone or in combination with 13-cis-RA show a significant increase of tumor macrophages infiltration compared to untreated mice. (E) Tumor macrophage infiltration in (D) was associated with both CD47 and SIRPα expression. Left panel shows the % of CD47-positive tumor cells (empty column) and the % of SIRPα-positive macrophages (black column) in tumors isolated from mice in (A) Representative flow cytometric histogram of CD47 staining for untreated mice and 8B6-treated treated mice on the bottom left. The bottom right indicates the representative flow cytometric of SIRPα-positive tumor-infiltrating macrophages for the untreated mice and mice treated with 13-cis-RA plus 8B6 mAb. Antibody 8B6 alone or in combination with 13-cis-RA increases both the % of CD47-expressing tumor cells and the % of SIRPα-positive macrophages in tumors. (F-G) Macrophages depletion using clodronate liposome impairs efficiency of 8B6 mAb treatment. Mice (6 animal/group) challenged with NXS2 cells as in (A) were received an administration of clodronate liposomes or liposomes by i.p. injection on days 4, 0, 3, 7, 10, 14, 17, and 21. Antibody 8B6 regime was given as in (A). Mice were euthanized on day 27 days for anti-tumor protection as in (A). (F) Clodronate treatment diminishes mAb 8B6 therapy efficiency compared to control 8B6-treated mice. (G) The effectiveness of macrophage depletion was determined by flow cytometry analysis of CD45+F4/80+CD11b+Ly6G-Ly6Clow-int cells among splenocytes from either the liposomes+8B6-treated mice or the clodronate liposomes+8B6-treated animals. Clodronate liposomes induced macrophage depletion relative to control mice treated with liposomes. Data represent mean±SEM (*p<0.5; **p<0.01; ***p<0.001; Mann-Whitney test).

FIG. 2. Both O-acetyl GD2-specific monoclonal antibody 8B6 and 13-cis-RA exposures induce CD47 phagocytosis checkpoint up-regulation in NB cells in vitro. The NB cells IMR5, LAN1 and NXS2 were respectively incubated for 72 hours with (A) 13-cis-RA, (B) mAb 8B6, and (C) 13-cis-RA plus mAb 8B6, as indicated. After incubation, CD47 cell surface expression was analyzed by flow cytometry analysis. Each therapy regime induces up-regulation of CD47 for all tested cell lines with the exception of NXS2 cells treated with mAb 8B6. Data are presented as the mean±SEM (*p<0.5; **p<0.01; ***p<0.001; Mann-Whitney test).

FIG. 3. Monoclonal antibody P84 against SIRPα that blocks CD47-SIRPα interaction enables OAcGD2-specific mAb 8B6-mediated antibody dependent cellular phagocytosis of macrophages towards OAcGD2-positive NXS2 NB cells. (A) In vitro differentiated macrophages were analyzed for expression of SIRPα and cultured NXS2 tumor cells were analyzed for expression of OAcGD2 and CD47. Left panel, representative flow cytometry histograms of macrophages stained with either control antibody or P84, as indicated; center panel, representative flow cytometry histogram of NXS2 tumor cells stained with control antibody or 8B6 as indicated; right panel, representative flow cytometry histogram of NXS2 tumor cells stained with control antibody or anti-CD47 mAb as indicated; (B-D) Flow cytometry analysis to determine ADCP activity of bone marrow-derived macrophages against NXS2 cells. (B) Macrophages were gated on F4/80 protein expression, NB cells were gated on eFluor 670 fluorescence. Phagocytosis was determined by increased eFluor 670 fluorescence of F4/80-expressing cells. The upper left plot shows representative flow cytometric histograms of phagocytosis assay performed with anti-SIRPα P84 mAb-opsonized macrophages; the upper center plot shows representative flow cytometric histograms of phagocytosis assay performed with 8B6-opsonized NXS2 cells, the upper left plot shows representative flow cytometric histograms of phagocytosis assay performed with P84-opsonized macrophages and 8B6-opsonized NXS2 cells. Lower panels, the macrophages were incubated with the control unopsonized Neuro 2A target cells as indicated. (C-D) The data shown are pooled from three independent experiments. All data points were collected in triplicate, and the mean % of phagocytosis±SEM were plotted against each experimental condition as indicated. Incubation with SIRPα blocking P84 antibody resulted in increased phagocytosis of 8B6-opsonized NXS2 cells (ns; not significant; *p<0.05; **p<0.01; Mann-Whitney test).

FIG. 4. SIRPα-specific mAb P84 enhances anti-OAcGD2 mAb 8B6 inhibition of NXS2 NB liver metastasis. (A) Mice (12 animal/group) were inoculated i.v. with 2.5×10⁵ NXS2 cells on day 0. Antibody 8B6 treatment was given i.v. on days 3, 7, 10, 14, 17, and 21. 13-cis-retinoic acid (10 mg/kg) was given per os daily on day 17 to 21. Antibody P84 (200 μg/mice) was given i.p. following the same 8B6-therapy schedule. Mice were euthanized on day 27 for treatment efficacy analysis. (B) Anti-tumor efficacy was evaluated by determining the liver weight on the fresh specimen. The y-axis starts at 0.8 g corresponding to the average of normal liver weight. Mean of liver weight±SEM of each treatment group, as depicted. SIRPα blockade increases the efficiency of the combination of 13-cis-RA with mAb 8B6 in vivo. (C) The mice body weight was recorded for each treatment group, as indicated. Mean weight of mice on day 0 was defined as 100% weight. Weight in each group remained stable for the period of the treatment (*p<0.05; **p<0.01; Mann-Whitney test).

TABLE OF SEQUENCES:

| SEQ | | |
|---|---|---|
| ID | Sequence | |
| NO | function | Sequence |
| 1 | 8B6 mAb VH- | EFTFTDYY |
| | CDR1 | |
| 2 | 8B6 mAb VH- | IRNRANGYTT |
| | CDR2 | |
| 3 | 8B6 mAb VH- | ARVSNWAFDY |
| | CDR3 | |
| 4 | 8B6 mAb VL- | QSLLKNNGNTFL |
| | CDR1 | |
| 5 | 8B6 mAb VL- | KVS |
| | CDR2 | |
| 6 | 8B6 mAb VL- | SQSTHIPYT |
| | CDR3 | |
| 7 | Humanized | D/EV/IVMTQSPL/AS/TLP/SV/L/AS/TL/P/VGD/Q/EQ/P/RA/VS/TI/LS/TCRS/AS |
| | anti-OAcGD2 | QSL/VL/VKN/SN/QG/A/SN/Y/ST/N/SF/YLH/N/S/A/Y/GWY/FL/QQK/RPGQ/KS/ |
| | VL consensus | A/VPK/Q/RL/R/VLIYK/G/LV/A/GSN/TRL/D/AS/TGV/IPD/A/SRFSGSGSGTY/D |
| | sequence | FTLK/TIS/NR/SV/LE/QA/PEDL/V/FG/AV/TYF/YCS/M/QQS/AT/YH/Q/NI/T/QP/ |
| | | SYTFGG/QGTKVEIK |
| 8 | Humanized | E/QVQLV/LESGGGLVQ/KPGG/RSLRLSCA/TT/ASE/GFTFT/S/GDY/HYMT/H/ |
| | anti-OAcGD2 | N/SWV/IRQAPGKGLEWL/VG/SF/YI/TRNR/K/SA/SN/SG/A/SY/GT/IT/IE/YYN/ |
| | VH consensus | AP/A/DSVKGRFTISRDN/GS/AKS/NI/S/TL/T/AYLQMNSLR/K/QT/AEDTAV/I/ |
| | sequence | LYYCA/TRVSNWA/YFDYWGQGTT/LL/VTVSS |
| 9 | Anti-OAcGD2 | EVQLVESGGGLVQPGRSLRLSCTTSEFTFTDYYMTWVRQAPGKGLEWLGFI |
| | VH49A | RNRANGYTTEYNPSVKGRFTISRDNSKSILYLQMNSLKTEDTAVYYCARVS |
| | | NWAFDYWGQGTLVTVSS |
| 10 | Anti-OAcGD2 | EVQLVESGGGLVQPGGSLRLSCATSEFTFTDYYMTWVRQAPGKGLEWLGFI |
| | VH72A | RNRANGYTTEYNPSVKGRFTISRDNSKNSLYLQMNSLKTEDTAVYYCARVS |
| | | NWAFDYWGQGTLVTVSS |
| 11 | Anti-OAcGD2 | EVQLVESGGGLVQPGRSLRLSCTTSEFTFTDYYMTWVRQAPGKGLEWLGFI |
| | VH49BHS | RNKANGYTTEYNPSVKGRFTISRDNSKSILYLQMNSLKTEDTAVYYCARVS |
| | | NWAFDYWGQGTLVTVSS |
| 12 | Anti-OAcGD2 | EVQLVESGGGLVQPGGSLRLSCATSEFTFSDYYMTWVRQAPGKGLEWLGFI |
| | VH72BHNPS | RNKANGYTTEYNPSVKGRFTISRDNSKNSLYLQMNSLKTEDTAVYYCARVS |
| | | NWAFDYWGQGTLVTVSS |
| 13 | Anti-OAcGD2 | DVVMTQSPLSLPVTLGQPASISCRSSQSLLKNNGNTFLHWYQQRPGQSPRLLI |
| | VL30A | YKVSNRLSGVPDRFSGSGSGTDFTLKISRVEAEDVGVYFCSQSTHIPYTFGGG |
| | | TKVEIK |
| 14 | Anti-OAcGD2 | DVVMTQSPLSLPVTPGEPASISCRSSQSLLKNNGNTFLHWYLQKPGQSPQLLI |
| | VL28A | YKVSNRLSGVPDRFSGSGSGTDFTLKISRVEAEDVGVYFCSQSTHIPYTFGQG |
| | | TKVEIK |
| 15 | Anti-OAcGD2 | DVVMTQSPLSLPVTPGEPASISCRSSQSLLKSNANTFLHWYLQKPGQSPQLLI |
| | VL28Bs01/A2 | YKVSNRLSGVPDRFSGSGSGTDFTLKISRVEAEDVGVYYCMQSTHIPYTFGQ |
| | | GTKVEIK |
| 16 | Anti-OAcGD2 | EVQLVESGGGLVQPGRSLRLSCTTSEFTFTDYYMTWVRQAPGKGLEWLGFI |
| | VH49BHSs | RNKASGYTTEYNPSVKGRFTISRDNSKSILYLQMNSLKTEDTAVYYCARVSN |
| | | WAFDYWGQGTLVTVSS |
| 17 | Anti-OAcGD2 | EVQLVESGGGLVQPGGSLRLSCATSEFTFSDYYMTWVRQAPGKGLEWLGFI |
| | VH72BHNPSs | RNKANSYTTEYNPSVKGRFTISRDNSKNSLYLQMNSLKTEDTAVYYCARVS |
| | | NWAFDYWGQGTLVTVSS |
| 18 | Anti-OAcGD2 | DVVMTQSPLSLPVTLGQPASISCRSSQSLLKNQGNTFLHWYQQRPGQSPRLLI |
| | VL30As | YKVSNRLSGVPDRFSGSGSGTDFTLKISRVEAEDVGVYFCSQSTHIPYTFGGG |
| | | TKVEIK |
| 19 | Anti-OAcGD2 | DVVMTQSPLSLPVTPGEPASISCRSSQSLLKSNGNTFLHWYLQKPGQSPQLLI |
| | VL28B01/A2 | YKVSNRLSGVPDRFSGSGSGTDFTLKISRVEAEDVGVYYCMQSTHIPYTFGQ |
| | | GTKVEIK |

### EXAMPLE

A. Efficacy of Therapeutic Combination of Anti-OAcGD2 Antibody (Optionally with Retinoic Acid) and Anti-SIRP-Alpha Antibody in an Immune Competent Mouse Model of Neuroblastoma

### Material & Methods

### Therapeutic Antibodies and Other Experimental Reagents

Anti-OAcGD2 mAb 8B6 (mouse IgG2a) and an isotype control mouse IgG2a mAb generated and purified as described earlier (24). Anti-SIRPα mAb P84 (rat IgG2a) and isotype control (rat IgG2a) were generously provided by OSE Immunotherapeutics (Nantes, France) (25). A BV421-conjugated mAb specific for mouse CD45 (clone 30F11), a FITC-conjugated mAb specific for mouse CD11b (clone M1/70), a PE-conjugated mAb specific for mouse F4/80 (clone T45-2342), a PerCP-Cy5.5-conjugated mAb specific for mouse Ly6C (clone AL-21) were from BD Bioscience (Franklin Lakes, NJ, USA). A BV421-conjugated mAb mouse IgG (clone R35-38), a FITC-conjugated mAb mouse IgG (clone A95-1), a PE-conjugated mAb mouse IgG (clone R35-95) and a PerCP-Cy5.5-conjugated mAb mouse IgG (clone R4-22) were from BD Bioscience (Franklin Lakes, NJ, USA). An APC-conjugated mAb against mouse Ly6G (clone REA525) and an APC-conjugated mAb against mouse IgG were from Miltenyi (Bergisch Gladbach, Germany). A PerCP-Cy5.5-conjugated mAb specific for mouse CD47 (clone miap 301) and a PerCP-Cy5.5 mAb mouse IgG (clone RTK2758) were from Biolegend (San Diego, CA, USA). A FITC-conjugated polyclonal antibody against mouse IgG was from Jackson Immunoresearch (Soham, UK) was used as a secondary antibody for 8B6 binding detection. An APC-conjugated polyclonal antibody (A10540) ThermoFisher Scientific (San Diego, CA, USA) was used as a secondary antibody for SIRPα binding. A mAb specific for CD16/CD32 (clone 93) was from Invitrogen (San Diego, CA, USA). Isotretinoin—13-cis-retinoic acid—was purchased from Sigma Aldrich (Saint Louis, MO, USA).

### Cell Culture

The human neuroblastoma IMR5 cell line was generously provided by Dr. Santos Susin (Inserm U.872, Paris, France). Mouse neuroblastoma NXS2 cell line was given to us by Dr. H. N. Lode (Universitätsklinikum Greifswald, Greifswald, Germany). Human LAN1 neuroblastoma cell line was obtained from the Children's Oncology Group Cell Culture and Xenograft Repository (Philadelphia, PA, USA). NXS2 cells were grown in DMEM with 10% heat-inactivated fetal calf serum, 2 mM L-Glutamin, 100 units/mL penicillin, and 100 μg/mL streptomycin, at 37° C. in 5% CO2. IMR5, LAN1 cells were grown in RPMI 1640 with 10% heat-inactivated fetal calf serum, 2 mM L-Glutamin, 100 units/mL penicillin and 100 μg/mL streptomycin, at 37° ° C. in 5% CO2.

### Cell Preparation and Flow Cytometry Analysis

Liver metastasis or spleen were dissociated with the mouse tumor dissociation kit and the gentle MACS dissociator (Miltenyi) according to the manufacturer's instructions. Single cell suspensions were obtained by passing through a 70 μm pore-size cell MACS SmartStrainer (Miltenyi). Red blood cells in the filtrate were lysed with RBCs lysis buffer (Stemcell, Vancouver, BC, Canada). The remaining cells were washed twice with PBS and, thereafter, stained with a Viobility 405/520 Fixable Dye stain (Miltenyi) to exclude dead cells.

For flow cytometry analysis, cells were first incubated with a mAb specific for mouse CD16/CD32 to prevent nonspecific binding of primary antibodies against FcgammaR, and then, were labeled with specific labeled mAbs. Separate experiments were performed with appropriate isotype-control antibodies. Quantification of total cell numbers by flow cytometry was performed done using fluorescent beads (C36950, Thermo Fisher, Waltham, MA, USA). Flow cytometric analyses were performed using an BD FACSCanto II (BD Bioscience) and data were analyzed using FlowJo software (Flowjo LLC, Oregon, OR, USA). The fold increase of tumor macrophage infiltration was calculated by dividing the number of tumor macrophage of each mice group by the mean of the number of tumor macrophage of untreated mice.

For determination of OAcGD2 expression on tumor cell lines, cells were incubated with either mAb 8B6 or isotype control, washed with PBS, and then incubated with FITC-conjugated secondary antibody. Stained cells were subjected to flow cytometry, and data were analyzed with FlowJo software.

### Depletion of Macrophages

Depletion of macrophages in 8-week-old A/J mice was performed clodronate liposomes or liposomes obtained from Liposoma (Liposoma BV, Amsterdam, the Netherlands) according to the manufacturer's instructions. Mice were given either clodronate liposomes or liposome i.p. 4 days before tumor cell injection and thereafter on days 0, 3, 7, 10, 14, 17, and 21 post-tumor cell inoculation. The effectiveness of macrophage depletion was determined by flow cytometry analysis of CD45+F4/80+CD11b+Ly6G-Ly6Clow-int cells among splenocytes from the treated animals.

### ADCP Assay

For preparation of bone marrow-derived mouse macrophages (BMDM), bone marrow cells were isolated from the femur and tibia of mice using a syringe fitted with a 27-gauge needle as describe previously with minor modifications (26). Bone marrow cells were (0.5×10⁶/mL) seeded on culture plate in RPMI 1640 medium supplemented with 25 mM Hepes, recombinant murine macrophage m-CSF (10 μg/mL, Bio-Techne, Minneapolis, MN, USA) and FCS 10%. For ADCP assays by flow cytometry as described previously with minor modifications (27), BMDM were plated at a density of 2×10⁴ cells per well in ultra-low attachment round-bottom 96-well plates. NXS2 target cells were labeled with eFluor 670 (Thermo Fisher) according to manufacturer instructions, added to the BMDM (effector cells), and incubated for 2 hours in the presence of OAcGD2-specific mAb 8B6. Alternatively, effector cells were pre-incubated with BMDMs with SIRPα-specific mAb P84 for 2 hours at 37° C. We used a tumor cell to macrophage ratio (E:T) of 2:1. Cells were then harvested, stained for F4/80-PE and analyzed by flow cytometry. The percentage of phagocytosis by BMDMs was calculated as: 100×F4/80+eFluor670+ cells/F4/80+eFluor670++F4/80+eFluor670-cells.

### Tumor Cell Engraftment and Treatments

NXS2 Cells (2.5×10⁵ in 100 μL of PBS) were transplanted into syngeneic 8-weeks old female A/J mice (Harlan Laboratories, Gannat, France) i.v., to induces liver metastasis 6. Mice received either mAb 8B6 (25 μg, i.v.) or mAb P84 (200 μg, i.p.) twice a week for 3 consecutive weeks beginning 3 days after tumor cell injection. Isotretinoin was given orally diluted in Ora-Plus® at 10 mg/kg daily for 5 consecutive days, starting week 3 after tumor cell challenge. Mice were euthanized on day 27 post-tumor challenge. Anti-tumor efficacy was evaluated by liver weight of the fresh specimen as described previously 6.

### Statistical Analysis

Statistical analysis was performed using Prism software (GraphPad, San Diego, CA, USA). Differences between untreated and treated groups in the in vitro experiences were analyzed by Mann-Whitney test. Statistical significance of liver weights and metastasis number of experimental groups of mice was tested by two-tailed Student's t test. A p value of less than 0.05 was considered to be statistically significant. All experiment results were shown as mean±standard error of the mean (SEM).

### Study Approval

All in vivo experiments were carried out in strict accordance with the recommendations in the Guide for the Care and Use of Laboratory Animals of the French Department of Agriculture (agreement number: 00186.02). The protocol was approved by the Committee on the Ethics of Animal Experiments of the Région Pays de la Loire (permit number: 03479.01). Mice were housed at the UTE-UN animal facility (Nantes, France).

### Results

### Macrophages Contributes to the Anti-Neuroblastoma Activity of OAcGD2-Specific Antibodies

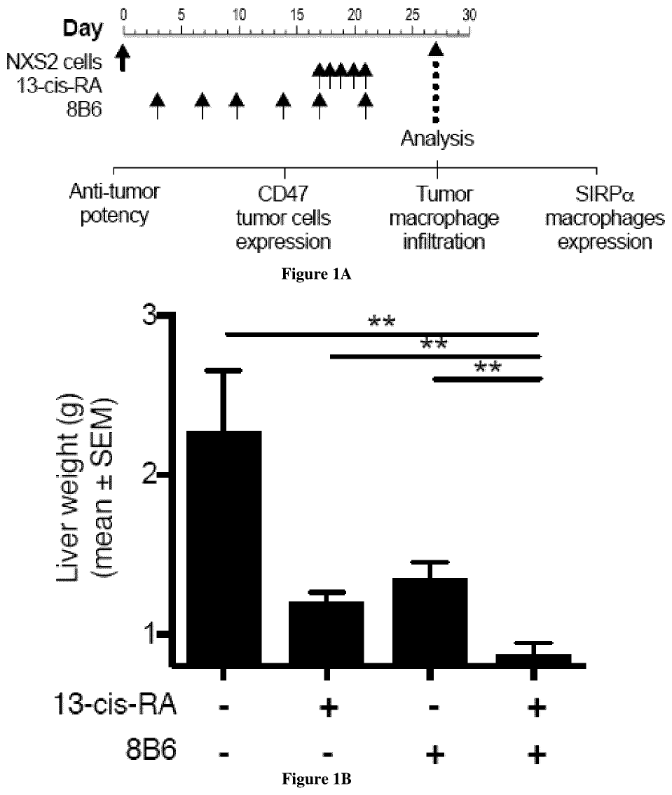

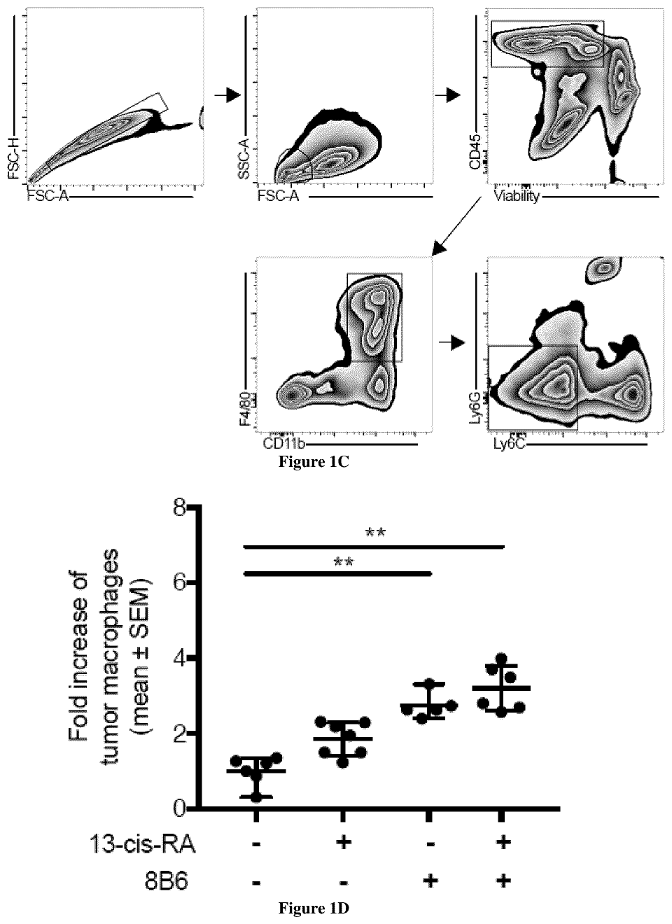

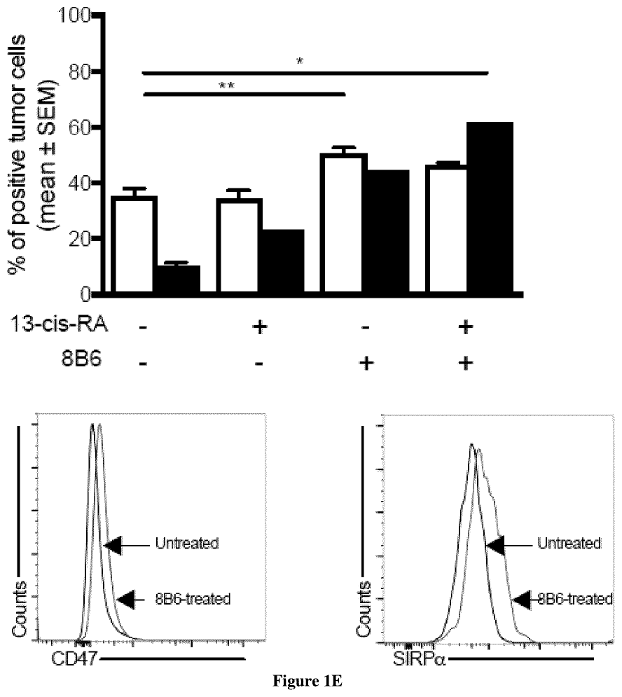

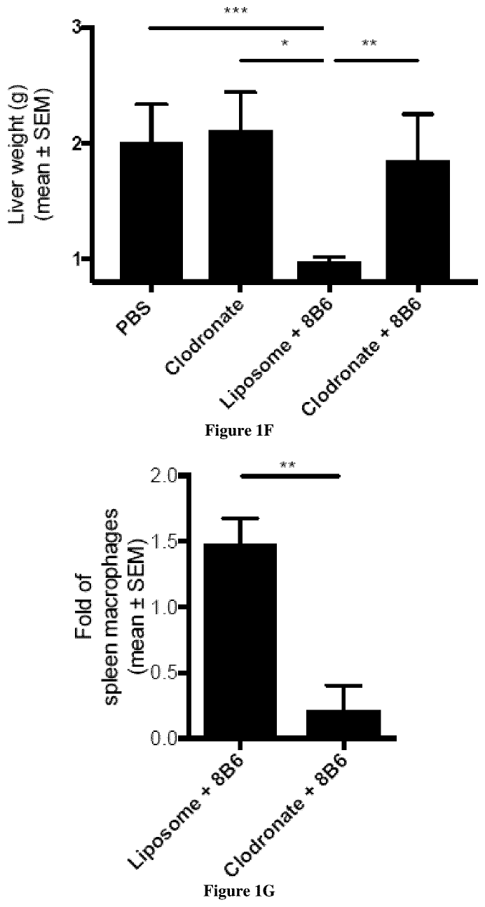

We first tested if macrophages were effector cells recruited by OAcGD2-specific mAb 8B6 in an immunocompetent context. We used the syngeneic model of NXS2 NB liver metastasis in A/J mice earlier to evaluate the anti-NB activity of mAb 8B6 used as a single agent 21. Immunotherapy with GD2-specific mAb in patient with NB is given, however, in combination with 13-cis-RA 4, 5. Therefore, we evaluated here the anti-NB effect of mAb 8B6 in combination with 13-cis-RA (FIG. 1). We calculated the 13-cis-RA dosage to reflect the human equivalent dose (22). Three days after i.v. NSX2 tumor cells inoculation, we assigned eight mice to treatment with either the single agent therapy, or the combination regimen. On day 27 after tumor cells inoculation, we determined the liver weight after mice euthanasia (FIG. 1A). The dose of 13-cis-RA (10 mg/kg, per os, days 17-22) that we used here, yielded a significant reduction of the tumor-related liver weight compared to the vehicle treated mice (13-cis-RA-treated group: 1.19±0.0 g; vehicle-treated group: 2.27±0.38 g, p<0.01, FIG. 1B). Antibody 8B6 cooperated with 13-cis-RA, resulting in a significant reduction of the liver weight compared to either 13-cis-RA, or mAb 8B6 used as single agent (1.35±0.10 g, p<0.01, FIG. 1B). The specificity of mAb 8B6 therapy was demonstrated, since treatment with an equivalent amount of non-specific antibody was completely ineffective (p>0.05 compared to vehicle-treated mice, FIG. 1B).

We next collected the residual tumors of these mice to examine whether tumor infiltrating macrophages were presents. We detected the presence of intra-tumor macrophages by flow cytometry analysis of CD45+F4/80+CD11b+Ly6G-Ly6Clow-int cells among single cell suspension prepared form resected tumors (FIG. 1C). Strikingly, we found that 8B6 mAb therapy, given either as single agent or in combination with 13-cis-RA, increased the level of tumor-infiltrating macrophages (8B6-treated group: 2.77±0.17, 8B6+13-cis-RA-treated group: 3.21±0.24, p<0.01, FIG. 1D) compared the untreated mice (1.00±0.16 g, p<0.05, FIG. 1D).

We went on to study using flow cytometry both CD47-NB cell surface expression and SIRPα-macrophage surface expression in these single cell suspensions. Earlier reports have shown that interaction between CD47, expressed broadly on normal and tumor cells, and the myeloid inhibitory receptor of phagocytosis SIRPα negatively regulate macrophage-mediated ADCP induced by anti-cancer therapeutic antibody 14, 15. Remarkably, we observed that mAb 8B6 monotherapy, or combined with 13-cis-RA, induced cell-surface CD47 up-regulation on NXS2 tumor cells compared to untreated mice (8B6 monotherapy: 50.56±2.16%; 8B6+13-cis-RA-treated mice: 46.33±1.09%; untreated mice: 35.19±2.93%; p<0.05; FIG. 1E). These results further correlated with a SIRPα up-regulation on tumor infiltrating macrophages (8B6-treated mice: 44.30±0.2%; 8B6+13-cis-RA-treated mice: 61.8±0.1%; untreated mice: 10.37±0.5%, p<0.5; FIG. 1E).

Taken together, these finding suggested a functional role of macrophage in 8B6 antitumor effect. Thus, we performed macrophage depletion in A/J mice and evaluated the anti-NB activity of mAb 8B6 (FIG. 1F-G). Using clodronate treatment, we achieved significant macrophage depletion in mice (clodronate-treated group: 2.11±0.33%, untreated group: 2.12±0.36%; p<0.01; FIG. 1F). Importantly, macrophage depletion resulted in a significant decreased of 8B6 therapeutic efficacy (macrophage-depleted mice: 0.22±0.18; 8B6-treated mice: 1.48±0.19; p<0.05; FIG. 1G).

### OAcGD2-Specific Monoclonal Antibody and 13-Cis-RA Exposure Induce CD47 Up-Regulation in NB Cells In Vitro.

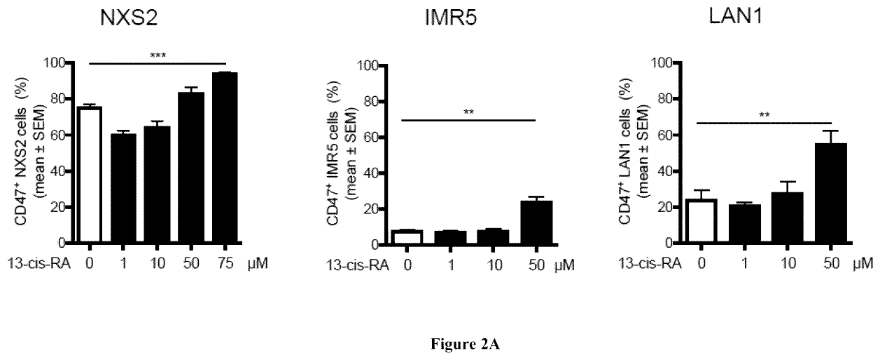

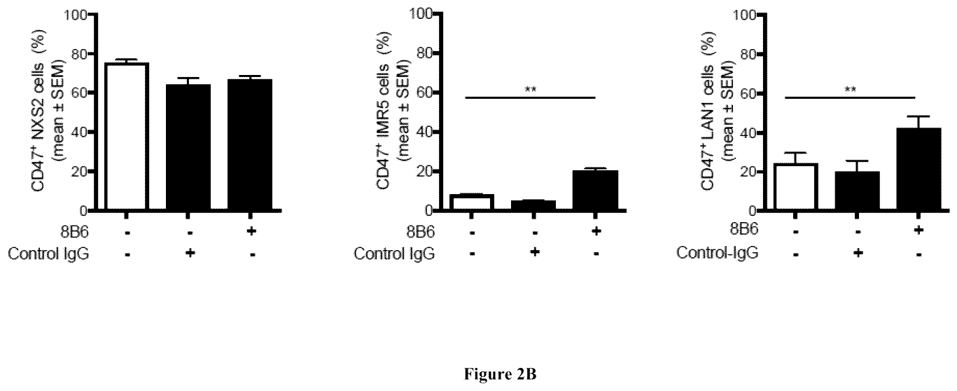

We next interrogate the impact of mAb 8B6, used alone or in combination with 13-cis-RA, on CD47 cell-surface expression in human NB cell line. After 72 hours incubation, cells were harvested, stained with a CD47-specific mAb and then subjected to flow cytometry analysis. Remarkably, we observed that both agents used alone or in combination induced CD47 up-regulation on the tested human NB cell lines with the exception of the NXS2 cells; these cells expressed, surprisingly, constitutively high CD47 level in vitro (FIG. 2A-C). The tested treatments had little impact on CD47 NXS2 cell-surface expression (FIG. 2A-C).

### Targeting CD47-SIRPα Interaction Enables 8B6-Mediated Antibody-Dependent Cellular Phagocytosis Against Neuroblastoma Cells

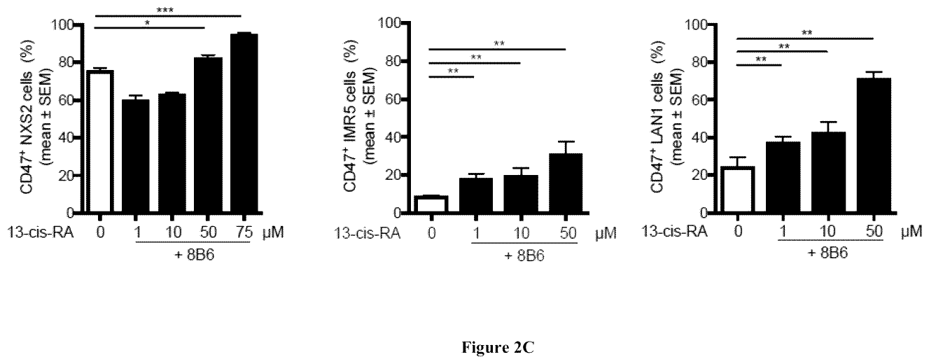

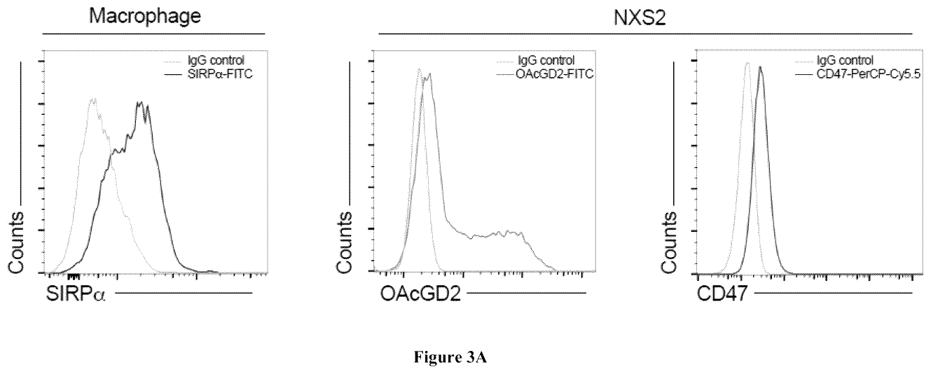

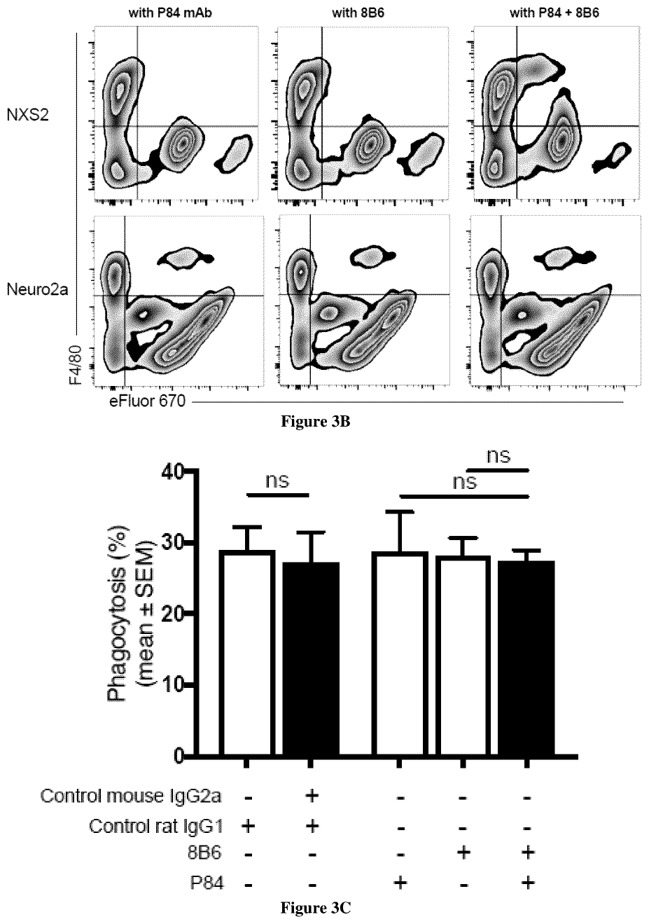

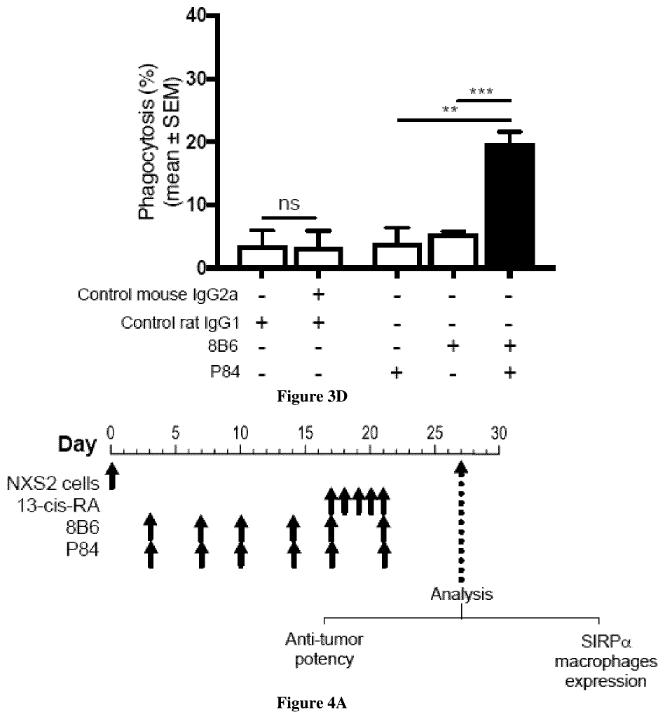

Over-expression of CD47 allows cancer cells to evade from macrophages for phagocytosis 13. Thus, we interrogated whether CD47 expression in NB cells would impair OAcGD2-specific mAb-mediated ADCP. We thus established an in vitro ADCP assay using OAcGD2+CD47+ NXS2 cells as target cells and bone marrow-derived macrophages as effector cells. A/J mouse bone marrow-derived macrophages obtained from A/J mice expressed SIRPα receptor (FIG. 3A). ADCP was analyzed by flow cytometry analysis; the gating strategy is depicted in FIG. 3B. OAcGD2+CD47+ NXS2 cells were labeled with eFluor 670 and opsonized with mAb 8B6. Target cells were added at a 2:1 target to effector cell ratio. F4/80+ macrophages were then analyzed for eFluor 670 fluorescence. When tested against NXS2 cells, bone marrow-derived macrophages were not enable to engulf 8B6-opsonized tumor cells (FIG. 3B-C). However, significant 8B6-mediated ADCP by bone marrow-derived macrophages occurred upon blockade of CD47-SIRPα interaction with a SIRPα-specific mAb (FIG. 3B-C). In the absence of mAb 8B6, we did not observe any tumor cells engulfment performed by macrophages. This result suggests that CD47-SIRPα interactions do not control antibody-independent phagocytosis mechanisms (FIG. 3B-C). Similarly, no increase of the magnitude of phagocytosis was observed in the OAcGD2-Neuro 2A cells (FIG. 3C-D). These results thus suggest that blockade of the immune checkpoint CD47-SIRPα pathway enables 8B6-mediated ADCP of NB cells by macrophages.

### SIRPα-Specific Blocking Antibody Potentiates OAcGD2-Specific Monoclonal Antibody Therapeutic Effects In Vivo

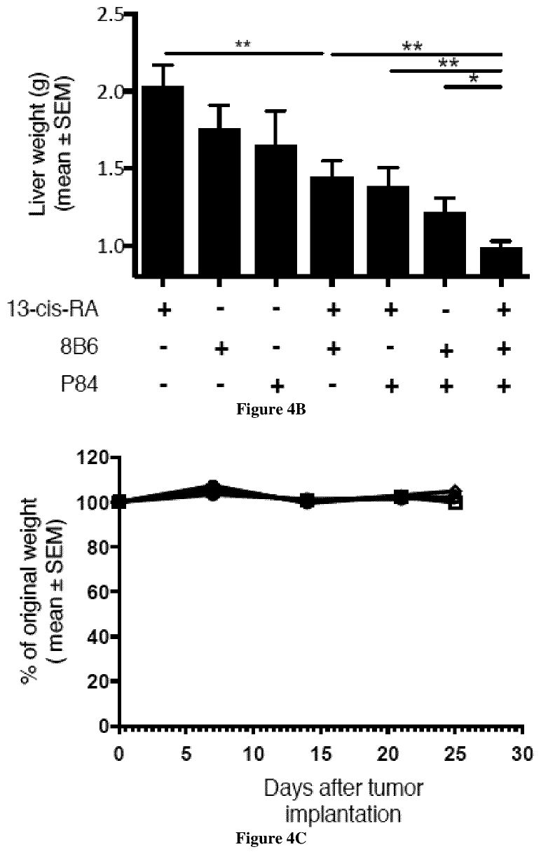

On the basis of the above results, we sought to determine whether SIRPα blockade could enhance the protective effect of 13-cis-RA+8B6 combination therapy in vivo. After NXS2 challenge, we randomized mice (n=5) to receive i.p. injections of saline, 13-cis-RA (10 mg/kg, per os, on days 17-21), SIRPα-specific mAb P84 (20 μg/mice/injection, on days 3, 7, 10, 14, 17, 21, i.p.), mAb 8B6 (25 μg/mice/injection, on days 3, 7, 10, 14, 17, 21, i.v.), anti-SIRPα mAb+13-cis-RA, mAb 8B6+13-cis-RA, or 13-cis-RA+mAb 8B6+anti-SIRPα mAb. Livers were harvested on day 27 and measurement of weight were achieved (FIG. 4A). Bitherapy with mAb 8B6 and mAb P84 significantly reduced the tumor-related liver weight compared to mice treated with 13-cis-RA, mAb 8B6, or with mAb P84 monotherapy (13-cis-RA: 1.95±0.12 g; mAb 8B6: 1.62±0.18 g; mAb P84: 1.65±0.22 g; p<0.05, FIG. 4B). However, the strongest therapeutic effect was observed in the group of mice treated with the tri-therapy (1.01±0.03 g, p<0.05, FIG. 4B). Mice that received the tri-therapy regimen did not show any significant changes in their body weight (FIG. 4C). These data demonstrate the beneficial effect of combining SIRPα blockade to the 13-cis-RA+8B6 regimen.

### B. Efficacy of Bispecific Antibody Targeting CD47 and O-Acetyl GD2 Ganglioside in an Immune Competent Mouse Model of Neuroblastoma

### Material & Methods

### Design, Manufacture & Quality Control of the Bispecific Antibodies

Anti-OAcGD2/CD47 bispecific antibody and control antibody are synthesized as asymmetric chimeric huIgG1 grafted with the anti-muCD47 A4 VHH (derived from camelid heavy chain only antibody) on one arm and the 8B6 humanized VH on the other arm. The anti-DOTA muVH (U.S. Pat. No. 7,230,085 B2) is used as negative control both sides. Within the bispecific antibody, H/H heterodimerization is enriched via “knob-in-hole” mutations and the H/L pairing will be directed by swapping CHI and CL domains in one arm (CrossMab technology). Control anti-OAcGD2, anti-CD47 huIgG1 and anti-DOTA huIgG1 are also synthesized. A mutation deleting protein-A binding is introduced in H chains with “holes” to eliminate mono-specific OAcGD2 antibodies.

Construct DNA is fully synthesized, cloned into pQMCF expression vector, transiently expressed in CHO cells, and purified using protein A affinity column.

### Quality Control

Purity is assessed via SDS-PAGE, analytical size-exclusion chromatography, and endotoxin tests.

Bispecific affinity is measured by BLI technology (Octet BLItZ) on immobilized purified muCD47 protein and OAcGD2.

### ADCP Assays

For preparation of bone marrow-derived mouse macrophages (BMDM), bone marrow cells are isolated from the femur and tibia of mice using a syringe fitted with a 27-gauge needle as describe previously with minor modifications. Bone marrow cells are (0.5×10⁶/mL) seeded on culture plate in RPMI 1640 medium supplemented with 25 mM Hepes, recombinant murine macrophage m-CSF (10 μg/mL, Bio-Techne, Minneapolis, MN, USA) and FCS 10%. For ADCP assays by flow cytometry as described previously with minor modifications, BMDM are plated at a density of 2×10⁴ cells per well in ultra-low attachment round-bottom 96-well plates. NXS2 target cells are labeled with eFluor 670 (Thermo Fisher) according to manufacturer instructions, added to the BMDM (effector cells), and incubated for 2 hours in the presence of OAcGD2-specific mAb 8B6. Alternatively, effector cells are pre-incubated with BMDMs with SIRP-specific mAb P84 for 2 hours at 37° C. We used a tumor cell to macrophage ratio (E:T) of 2:1. Cells are harvested, stained for F4/80-PE and analyzed by flow cytometry. The percentage of phagocytosis by BMDMs is calculated as: 100 F4/80+eFluor670+ cells/F4/80+eFluor670++F4/80+eFluor670− cells.

### Mice and Tumor Models

NXS2 Cells (2.5×10⁵ in 100 μL of PBS) are transplanted into syngeneic 8-weeks old female A/J mice (Harlan Laboratories, Gannat, France) i.v., to induce liver metastasis. Mice receive anti-OAcGD2 antibody (25 μg, i.v.) optionally with 13-cis-RA, CD47 blocking antibody (200 μg, i.p.), OAcGD2/CD47 bispecific antibody, alone or in combination according to the invention, twice a week for 3 consecutive weeks beginning 3 days after tumor cell injection. Mice are euthanized on day 27 post-tumor challenge. Anti-tumor efficacy is evaluated by liver weight of the fresh specimen. Experiments is repeated using control bispecific antibodies.

### Results

Strong anti-NB effect is observed in the mice treated with OAcGD2/CD47 bispecific antibody optionally in combination with 13-cis-RA.

### C. Anti-Tumoral Efficacy of Bispecific Antibody Targeting CD47 and O-Acetyl GD2 Ganglioside Assessed In Vitro in Human Cancer Models and In Vivo in an Immune-Competent Syngeneic Anti-Neuroblastoma Mouse Model

### Antibody Design and Qualification

### Design and Manufacture of the Bispecific Antibodies

Anti-OAcGD2/CD47 bispecific antibody was synthesized as an asymmetric huIgG1 with the humanized anti-OAcGD2 VH on one arm and the anti-mouse CD47 VHH on the other arm. The anti-DOTA muVH (patent U.S. Pat. No. 7,230,085 B2) is used as negative control in bispecific antibody constructs (Anti-DOTA/Anti-OAcGD2 and Anti-DOTA/Anti-CD47). Within the bispecific antibody constructs, H/H heterodimerization is enriched via “knob-in-hole” mutations and the H/L pairing is directed by swapping CHI and CL domains in one arm (CrossMab technology, Schaefer W. et al. PNAS 2011; 108(27):11187-92). Control monospecific antibodies (anti-OAcGD2, anti-CD47 and anti-DOTA) are synthesized using the same “knob-in-hole” structure. A mutation deleting protein-A binding is introduced in H chains with “holes” to eliminate mono-specific OAcGD2 antibodies. Construct DNA is fully synthesized, cloned into pQMCF expression vector, transiently expressed in CHO cells, and purified using protein A affinity column.

### Antibody Sequences

The sequences of the light and heavy chains of the monospecific and bispecific antibodies are as shown in the Table below:

| SEQ | | |
|---|---|---|
| ID | Sequence | |
| NO | function | Sequence |
| 20 | Anti-OAcGD2 | EVQLVESGGGLVQPGGSLRLSCATSEFTFSDYYMTWVRQAPGKGLEWLG |
| | HC | FIRNKANSYTTEYNPSVKGRFTISRDNSKNSLYLQMNSLKTEDTAVYYCA |
| | (anti-OAcGD2 | RVSNWAFDYWGQGTLVTVSSASTKGPSVFPLAPSSKSTSGGTAALGCLV |
| | anti-CD47 | KDYFPEPVTVSWNSGALTSGVHTFPAVLQSSGLYSLSSVVTVPSSSLGTQT |
| | bispecific) | YICNVNHKPSNTKVDKRVEPKSCDKTHTCPPCPAPELLGGPDVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPEEKTISKAKGQPR |
| | | EPQVYTLPPCREEMTKNQVSLWCLVKGFYPSDIAVEWESNGQPENNYKT |
| | | TPPVLDSDGSFFLYSKLTVDKSRWQQGNVFSCSVLHEALHSHYTQKSLSL |
| | | SPGK |
| 21 | Anti-OAcGD2 | DVVMTQSPLSLPVTLGQPASISCRSSQSLLKNNGNTFLHWYQQRPGQSPR |
| | LC | LLIYKVSNRLSGVPDRFSGSGSGTDFTLKISRVEAEDVGVYFCSQSTHIPYT |
| | (anti-OAcGD2 | FGGGTKVEIKRTVAAPSVFIFPPSDEQLKSGTASVVCLLNNFYPREAKVQ |
| | anti-CD47 | WKVDNALQSGNSQESVTEQDSKDSTYSLSSTLTLSKADYEKHKVYACEV |
| | bispecific) | THQGLSSPVTKSFNRGEC |
| 22 | Anti-CD47 VHH | QVQLVESGGGLVEPGGSLRLSCAASGIIFKINDMGWYRQAPGKRREWVA |
| | (anti-OAcGD2 | ASTGGDEAIYRDSVKDRFTISRDAKNSVFLQMNSLKPEDTAVYYCTAVIS |
| | anti-CD47 | TDRDGTEWRRYWGQGTQVTVSSGGGGGGSDKTHTCPPCPAPELLGGPD |
| | bispecific) | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPEEKTIS |
| | | KAKGQPREPQVCTLPPSREEMTKNQVSLSCAVKGFYPSDIAVEWESNGQP |
| | | ENNYKTTPPVLDSDGSFFLVSKLTVDKSRWQQGNVFSCSVLHEALHSRY |
| | | TQKSLSLSPGK |
| 23 | Anti-DOTA HC | EVMLVESGGGLVKPGGSLTLSCAASGFTFTAHAMSWVRQTPEKRLEWV |
| | (anti-DOTA anti- | ATISGGGTYTYFPDSFQGRFTISSDNAKNTLYLQMSSLRSEDTSMYFCTRH |
| | CD47 bispecific) | GDYRYAFGYWGQGTLVTVSAASTKGPSVFPLAPSSKSTSGGTAALGCLV |
| | | KDYFPEPVTVSWNSGALTSGVHTFPAVLQSSGLYSLSSVVTVPSSSLGTQT |
| | | YICNVNHKPSNTKVDKRVEPKSCDKTHTCPPCPAPELLGGPDVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPEEKTISKAKGQPR |
| | | EPQVYTLPPCREEMTKNQVSLWCLVKGFYPSDIAVEWESNGQPENNYKT |
| | | TPPVLDSDGSFFLYSKLTVDKSRWQQGNVFSCSVLHEALHSHYTQKSLSL |
| | | SPGK |
| 24 | Anti-DOTA LC | QAVTQESALTTSPGETVTLTCRSSSGAVTTNNYANWVQEKPDHLFTGLIG |
| | (anti-DOTA anti- | GTNNRAPGVPARFSGSLIGDKAALTITGAQTEDEAIYFCALWFSDHWVFG |
| | CD47 bispecific) | GGTELTVLGRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGEC |
| 25 | Anti-CD47 VHH | QVQLVESGGGLVEPGGSLRLSCAASGIIFKINDMGWYRQAPGKRREWVA |
| | (anti-DOTA anti- | ASTGGDEAIYRDSVKDRFTISRDAKNSVFLQMNSLKPEDTAVYYCTAVIS |
| | CD47 bispecific) | TDRDGTEWRRYWGQGTQVTVSSGGGGGGSDKTHTCPPCPAPELLGGPD |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPEEKTIS |
| | | KAKGQPREPQVCTLPPSREEMTKNQVSLSCAVKGFYPSDIAVEWESNGQP |
| | | ENNYKTTPPVLDSDGSFFLVSKLTVDKSRWQQGNVFSCSVLHEALHSRY |
| | | TQKSLSLSPGK |
| 26 | Anti-OAcGD2 | EVQLVESGGGLVQPGGSLRLSCATSEFTFSDYYMTWVRQAPGKGLEWLG |
| | HC | FIRNKANSYTTEYNPSVKGRFTISRDNSKNSLYLQMNSLKTEDTAVYYCA |
| | (anti-OAcGD2 | RVSNWAFDYWGQGTLVTVSSASTKGPSVFPLAPSSKSTSGGTAALGCLV |
| | anti-DOTA | KDYFPEPVTVSWNSGALTSGVHTFPAVLQSSGLYSLSSVVTVPSSSLGTQT |
| | bispecific) | YICNVNHKPSNTKVDKRVEPKSCDKTHTCPPCPAPELLGGPDVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPEEKTISKAKGQPR |
| | | EPQVYTLPPCREEMTKNQVSLWCLVKGFYPSDIAVEWESNGQPENNYKT |
| | | TPPVLDSDGSFFLYSKLTVDKSRWQQGNVFSCSVLHEALHSHYTQKSLSL |
| | | SPGK |
| 27 | Anti-OAcGD2 | DVVMTQSPLSLPVTLGQPASISCRSSQSLLKNNGNTFLHWYQQRPGQSPR |
| | LC | LLIYKVSNRLSGVPDRFSGSGSGTDFTLKISRVEAEDVGVYFCSQSTHIPYT |
| | (anti-OAcGD2 | FGGGTKVEIKRTVAAPSVFIFPPSDEQLKSGTASVVCLLNNFYPREAKVQ |
| | anti-DOTA | WKVDNALQSGNSQESVTEQDSKDSTYSLSSTLTLSKADYEKHKVYACEV |
| | bispecific) | THQGLSSPVTKSFNRGEC |
| 28 | Anti-DOTA HC | EVMLVESGGGLVKPGGSLTLSCAASGFTFTAHAMSWVRQTPEKRLEWV |
| | (anti-OAcGD2 | ATISGGGTYTYFPDSFQGRFTISSDNAKNTLYLQMSSLRSEDTSMYFCTRH |
| | anti-DOTA | GDYRYAFGYWGQGTLVTVSARTVAAPSVFIFPPSDEQLKSGTASVVCLL |
| | bispecific) | NNFYPREAKVQWKVDNALQSGNSQESVTEQDSKDSTYSLSSTLTLSKAD |
| | | YEKHKVYACEVTHQGLSSPVTKSFNRGECDKTHTCPPCPAPELLGGPDVF |
| | | LFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPEEKTISKA |
| | | KGQPREPQVCTLPPSREEMTKNQVSLSCAVKGFYPSDIAVEWESNGQPEN |
| | | NYKTTPPVLDSDGSFFLVSKLTVDKSRWQQGNVFSCSVLHEALHSRYTQ |
| | | KSLSLSPGK |
| 29 | Anti-DOTA LC | QAVTQESALTTSPGETVTLTCRSSSGAVTTNNYANWVQEKPDHLFTGLIG |
| | (anti-OAcGD2 | GTNNRAPGVPARFSGSLIGDKAALTITGAQTEDEAIYFCALWFSDHWVFG |
| | anti-DOTA | GGTELTVLGASTKGPSVFPLAPSSKSTSGGTAALGCLVKDYFPEPVTVSW |
| | bispecific) | NSGALTSGVHTFPAVLQSSGLYSLSSVVTVPSSSLGTQTYICNVNHKPSNT |
| | | KVDKRVEPKSC |
| 30 | Anti-OAcGD2 | EVQLVESGGGLVQPGGSLRLSCATSEFTFSDYYMTWVRQAPGKGLEWLG |
| | HC | FIRNKANSYTTEYNPSVKGRFTISRDNSKNSLYLQMNSLKTEDTAVYYCA |
| | (monospecific) | RVSNWAFDYWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQT |
| | | YICNVNHKPSNTKVDKRVEPKSCDKTHTCPPCPAPELLGGPDVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPEEKTISKAKGQPR |
| | | EPQVYTLPPSREEMTKNQVSLTCLVKGFYPSDIAVEWESNGQPENNYKTT |
| | | PPVLDSDGSFFLYSKLTVDKSRWQQGNVFSCSVLHEALHSHYTQKSLSLS |
| | | PGK |
| 31 | Anti-OAcGD2 | DVVMTQSPLSLPVTLGQPASISCRSSQSLLKNNGNTFLHWYQQRPGQSPR |
| | LC | LLIYKVSNRLSGVPDRFSGSGSGTDFTLKISRVEAEDVGVYFCSQSTHIPYT |
| | (monospecific) | FGGGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| 32 | Anti-DOTA HC | EVMLVESGGGLVKPGGSLTLSCAASGFTFTAHAMSWVRQTPEKRLEWV |
| | (monospecific) | ATISGGGTYTYFPDSFQGRFTISSDNAKNTLYLQMSSLRSEDTSMYFCTRH |
| | | GDYRYAFGYWGQGTLVTVSAASTKGPSVFPLAPSSKSTSGGTAALGCLV |
| | | KDYFPEPVTVSWNSGALTSGVHTFPAVLQSSGLYSLSSVVTVPSSSLGTQT |
| | | YICNVNHKPSNTKVDKRVEPKSCDKTHTCPPCPAPELLGGPDVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPEEKTISKAKGQPR |
| | | EPQVYTLPPSREEMTKNQVSLTCLVKGFYPSDIAVEWESNGQPENNYKTT |
| | | PPVLDSDGSFFLYSKLTVDKSRWQQGNVFSCSVLHEALHSHYTQKSLSLS |
| | | PGK |
| 33 | Anti-DOTA LC | QAVTQESALTTSPGETVTLTCRSSSGAVTTNNYANWVQEKPDHLFTGLIG |
| | (monospecific) | GTNNRAPGVPARFSGSLIGDKAALTITGAQTEDEAIYFCALWFSDHWVFG |
| | | GGTELTVLGRTVAAPSVFIFPPSDEQLKSGTASVVCLLNNFYPREAKVQW |
| | | KVDNALQSGNSQESVTEQDSKDSTYSLSSTLTLSKADYEKHKVYACEVT |
| | | HQGLSSPVTKSFNRGEC |
| 34 | Anti-CD47 HC | QVQLVESGGGLVEPGGSLRLSCAASGIIFKINDMGWYRQAPGKRREWVA |
| | (monospecific) | ASTGGDEAIYRDSVKDRFTISRDAKNSVFLQMNSLKPEDTAVYYCTAVIS |
| | | TDRDGTEWRRYWGQGTQVTVSSGGGGGGSDKTHTCPPCPAPELLGGPD |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPEEKTIS |
| | | KAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVLHEALHSHY |
| | | TQKSLSLSPGK |
| 35 | Anti-CD47 | GIIFKIND |
| | VHH-CDR1 | |
| 36 | Anti-CD47 | ASTGGDEA |
| | VHH-CDR2 | |
| 37 | Anti-CD47 | TAVISTDRDGTEWRR |
| | VHH-CDR3 | |
| 38 | Anti-CD47 | QVQLVESGGGLVEPGGSLRLSCAASGIIFKINDMGWYRQAPGKRREWVA |
| | VHH | ASTGGDEAIYRDSVKDRFTISRDAKNSVFLQMNSLKPEDTAVYYCTAVIS |
| | | TDRDGTEWRRYWGQGTQVTVSS |

### Quality Control of the Antibodies

Purity was assessed via SDS-PAGE, analytical size-exclusion chromatography, and endotoxin tests.

### Bispecific Binding Using BioLayer Interferometry (BLI)

Bispecific binding was evaluated by BLI technology (Octet BLItZ) on immobilized purified CD47 protein and OAcGD2. BLI analysis was performed on Octet R8 or R2 instrument using standard streptavidin (SA) biosensors or standard NiNTA biosensors. PBS was used as loading buffer. OAcGD2 sugar (OAcGD2s) loading was performed by loading biotinylated OAcGD2s (used at 0.25 μg/mL in PBS) to SA-biosensors for 60 seconds. CD47 loading was performed by loading His-tagged CD47 (used at 200 nM in PBS) to NiNTA-biosensors for 60 seconds. After loading, biosensors were washed in standard assay buffer (PBS-BSA 0.1%-Tween20 0.02%) until a stable baseline was obtained. Antibody binding was performed by moving OAcGD2s-loaded biosensors or CD47-loaded biosensors to wells containing antibody samples at the concentration of 100 nM in standard assay buffer. Then, the antibody-loaded biosensors were moved to standard assay buffer to examine antibody dissociation. Data were exported from the Octet Analysis Studio 12.2 software into excel files to confirm the binding of the antibodies to OAcGD2s or CD47 (No kinetics were derived from these experiments).

### In Vitro Studies

### Cell Culture

Mouse neuroblastoma NXS2 cell line was kindly provided by Dr. H. N. Lode (Universitätsklinikum Greifswald, Greifswald, Germany). NXS2 cells were grown in DMEM supplemented with 10% heat-inactivated fetal calf serum, 2 mM L-Glutamin, 100 units/mL penicillin, and 100 μg/mL streptomycin, at 37° C. in a humidified incubator at 5% CO2 atmosphere. Human neuroblastoma LAN-1 cell line (DSMZ GmbH, Germany) were grown in RPMI 1640 supplemented with 10% heat-inactivated fetal calf serum, 2 mM L-Glutamin, 100 units/mL penicillin, and 100 μg/mL streptomycin, at 37° C. in a humidified incubator at 5% CO2 atmosphere.

### ADCP Assay

Mouse macrophages were obtained from 10- to 11-week-old A/J mice bone marrow. Briefly, mice were euthanized and femura and tibiae were isolated. The bones were kept in ice-cold PBS. By flushing them with PBS, bone marrow cells were gathered, and plated at 0.5×10⁶/mL in 6-well cell culture plate in mouse macrophage medium (RPMI1640 with FBS 10%, 1× penicillin/streptomycin, 2 mM glutamin, and 25 mM Hepes, all from Gibco) containing 10 μg/mL m-CSF (Biotechne). The medium was changed after 3 days and cells were harvested for experiments on days 7-10. NXS2 cells were labelled with eFluor 670 (ThermoFisher) according to manufacturer instructions and pre-treated with or without 10 μg/mL of Anti-OAcGD2 antibody. NXS2 labelled cells were incubated with bone marrow-derived macrophages in medium with or without prior addition of 0.2 μg/mL or 1 μg/mL anti-CD47 monoclonal antibody at 37° C. for 2 hours in ultra-low attachment round-bottom 96-well plates. The macrophage ratio tumor cell was 2:1 (E:T). Subsequently, co-cultures were stained with F4/80-FITC mAb and washed before analysis by flow cytometry. Analysis of cells were performed by using an BD LSRII cytometer (BD Biosciences, San Jose, CA, USA) and data were analyzed using FlowJo software (Flowjo LLC, Oregon, OR, USA). Phagocytic cell percentages were identified by double positivity for eFluor 670 and F4/80 and calculated as follows:

100×F4/80pos eFluor670pos cells/F4/80pos eFluor670pos+F4/80pos eFluor670neg cells.

### Calcein Release ADCC Assay

Human neuroblastoma LAN-1 cells were labelled with Calcein-AM for 30 min, then washed and plated at a density of 5000 cells/well onto U bottom 96-well plates. Varying concentrations of the OAcGD2/CD47 bispecific antibody and control monospecific antibodies were added and peripheral blood mononuclear cells (PBMCs) were added to the LAN-1 cells wells at 1×10⁵ cells/well and incubated for 2 h (an effector:target ratio of 20:1) at 37° C. The supernatants were analyzed using fluorometry (on a Fluostar Omega Reader) to measure calcein release (cell death). For maximal release, the cells were lysed with 2% Triton X-100. The fluorescence value of the culture medium background was subtracted from that of the experimental release (A), the target cell spontaneous release (B) and the target cell maximal release (C). The cytotoxicity and ADCC percentages for each plate (in triplicate) were calculated using the following formulas:

Cytotoxicity (%)=(A−B)/(C−B)×100

ADCC (%)=Cytotoxicity (%,with antibody)−Cytotoxicity (%,without antibody)

### ADCC Reporter Bioassay

This experiment was performed using Promega ADDC Reporter Bioassay kit (Ref. G7010). Human neuroblastoma LAN-1 cells were plated onto white 96-well plates at a density of 12 500 cells/well and allow to grow in fresh culture medium for 20 to 24 hours. Twenty-four hours later, medium was replaced by ADCC Bioassay buffer (RPMI 1640 containing low IgG serum). Series of concentrations for the OAcGD2/CD47 bispecific antibody and control monospecific antibodies ranging from 10 to 0 μg/ml were added. ADCC Bioassay effector cells (Jurkat cells stably expressing the FcγRIIIa receptor, V₁₅₈ and NFAT pathway quantified through firefly luciferase production) were then added to the LAN-1 cells wells at 7.5×10⁵ cells/well and incubated for 6 h (an effector:target ratio of 6:1) at 37° C. Six hours after, plates were allowed to equilibrate to ambient temperature (22-25° C.) during 15 minutes, and Bio-Glo™ Luciferase Assay Reagent was added to all wells and incubated at ambient temperature for 5-30 minutes. Luminescence was measured using a Fluostar Omega plate Reader. Luminescence value of the culture medium background was subtracted from that of the experimental Relative Light Units (RLU) values. Graph data were plotted as RLU versus Log10 [antibody concentration] and fit curves to calculate EC50 of antibodies were calculated using GraphPad Prism software.

### In Vivo Studies

Anti-neuroblastoma efficacy of anti-OAcGD2, anti-CD47 and anti-OAcGD2/Anti-CD47 bispecific antibody treatments was determined in the murine NXS2 neuroblastoma experimental liver metastasis model in A/J mice.

### Anti-Tumoral Efficacy Study of Repeated Doses

Animals: 10-weeks old female A/J mice (Jackson Laboratories, US). Ten mice were assigned per group of treatment. Mice were housed at the UTE-IRS2 animal facility (Nantes, France). All in vivo experiments were carried out in strict accordance with the recommendations in the Guide for the Care and Use of Laboratory Animals of the French Department of Agriculture. The protocol was approved by the Committee on the Ethics of Animal Experiments of the Région Pays de la Loire (permit number: 35513v4).

Engraftment: NXS2 Cells (2.5×10⁵ cells in 100 μL of non-supplemented DMEM medium) were transplanted i.v. into syngeneic 10-weeks old female A/J mice, to induce liver metastasis.

Treatment schedule: Mice received either anti-OAcGD2 antibody, or CD47 blocking antibody, or OAcGD2/CD47 bispecific antibody, or anti-DOTA mAb as isotypic control alone as single agent therapy or the combination regimen for anti-OAcGD2 and anti-CD47 antibodies twice a week for 3 consecutive weeks beginning 3 days after tumor cell engraftment. We assigned 10 mice to treatment with either the single agent therapy (50 μg per injection i.v.; 300 μg total dose injection), or the combination regimen for anti-OAcGD2 and anti-CD47 antibodies (25 μg for each mAb mixed per injection; 300 μg mAb total dose injection). Mice were euthanized on day 28 post-tumor challenge, and anti-tumor efficacy was evaluated by determining the liver weight of fresh specimen and the number of liver metastasis.

### Dose-Response Study

Animals: 10-weeks old female A/J mice (Jackson Laboratories, US). Ten mice were assigned per group of treatment. Mice were housed at the UTE-IRS2 animal facility (Nantes, France). All in vivo experiments were carried out in strict accordance with the recommendations in the Guide for the Care and Use of Laboratory Animals of the French Department of Agriculture. The protocol was approved by the Committee on the Ethics of Animal Experiments of the Région Pays de la Loire (permit number: 35513v4).

Engraftment: NXS2 Cells (2.5×10⁵ cells in 100 μL of non-supplemented DMEM medium) were transplanted i.v. into syngeneic 10-weeks old female A/J mice, to induce liver metastasis.

Treatment schedule: The single agent therapy was evaluated in a dose response experiment with following protocol. Mice received either anti-OAcGD2 antibody, or OAcGD2/CD47 bispecific antibody, or anti-DOTA mAb as isotypic control at a dose of 50 μg per injection i.v. (300 μg total dose injection), 16.67 μg per injection i.v. (100 μg total dose injection), 5 μg per injection i.v. (30 μg total dose injection) or 1.67 μg per injection i.v. (10 μg total dose injection). We assigned 10 mice per group of treatment. Mice were euthanized on day 28 post-tumor challenge, and anti-tumor efficacy was evaluated by determining the liver weight of fresh specimen and the number of liver metastasis.

### REFERENCES

Throughout this application, various references describe the state of the art to which this invention pertains. The disclosures of these references are hereby incorporated by reference into the present disclosure.
1. Nakagawara A, Ohira M. Comprehensive genomics linking between neural development and cancer: neuroblastoma as a model. Cancer Lett 2004; 204: 213-24.
2. Katzenstein H M, Bowman L C, Brodeur G M, Thorner P S, Joshi V V, Smith E I, Look A T, Rowe S T, Nash M B, Holbrook T, Alvarado C, Rao P V, et al. Prognostic significance of age, MYCN oncogene amplification, tumor cell ploidy, and histology in 110 infants with stage D(S) neuroblastoma: the pediatric oncology group experience—a pediatric oncology group study. J Clin Oncol 1998; 16: 2007-17.
3. Cohn S L, Pearson A D, London W B, Monclair T, Ambros P F, Brodeur G M, Faldum A, Hero B, Iehara T, Machin D, Mosseri V, Simon T, et al. The International Neuroblastoma Risk Group (INRG) classification system: an INRG Task Force report. J Clin Oncol 2009; 27: 289-97.
4. Yu A L, Gilman A L, Ozkaynak M F, London W B, Kreissman S G, Chen H X, Smith M, Anderson B, Villablanca J G, Matthay K K, Shimada H, Grupp S A, et al. Anti-GD2 antibody with G M-CSF, interleukin-2, and isotretinoin for neuroblastoma. N Engl J Med 2010; 363: 1324-34.
5. Ladenstein R, Potschger U, Valteau-Couanet D, Luksch R, Castel V, Yaniv I, Laureys G, Brock P, Michon J M, Owens C, Trahair T, Chan G C F, et al. Interleukin 2 with anti-GD2 antibody ch14.18/CHO (dinutuximab beta) in patients with high-risk neuroblastoma (HR-NBL1/SIOPEN): a multicentre, randomised, phase 3 trial. Lancet Oncol 2018; 19: 1617-29.
6. Zeng Y, Fest S, Kunert R, Katinger H, Pistoia V, Michon J, Lewis G, Ladenstein R, Lode H N. Anti-neuroblastoma effect of ch14.18 antibody produced in CHO cells is mediated by NK-cells in mice. Mol Immunol 2005; 42: 1311-9.
7. Siebert N, Jensen C, Troschke-Meurer S, Zumpe M, Juttner M, Ehlert K, Kietz S, Muller I, Lode H N. Neuroblastoma patients with high-affinity FCGR2A, -3A and stimulatory KIR 2DS2 treated by long-term infusion of anti-GD2 antibody ch14.18/CHO show higher ADCC levels and improved event-free survival. Oncoimmunology 2016; 5: e1235108.
8. Tran H C, Wan Z, Sheard M A, Sun J, Jackson J R, Malvar J, Xu Y, Wang L, Sposto R, Kim E S, Asgharzadeh S, Seeger R C. TGFbetaR1 Blockade with Galunisertib (LY2157299) Enhances Anti-Neuroblastoma Activity of the Anti-GD2 Antibody Dinutuximab (ch14.18) with Natural Killer Cells. Clin Cancer Res 2017; 23: 804-13.
9. Kushner B H, Cheung N K. G M-CSF enhances 3F8 monoclonal antibody-dependent cellular cytotoxicity against human melanoma and neuroblastoma. Blood 1989; 73: 1936-41.
10. Cheung N K, Sowers R, Vickers A J, Cheung I Y, Kushner B H, Gorlick R. FCGR2A polymorphism is correlated with clinical outcome after immunotherapy of neuroblastoma with anti-GD2 antibody and granulocyte macrophage colony-stimulating factor. J Clin Oncol 2006; 24: 2885-90.
11. Raffaghello L, Marimpietri D, Pagnan G, Pastorino F, Cosimo E, Brignole C, Ponzoni M, Montaldo P G. Anti-GD2 monoclonal antibody immunotherapy: a promising strategy in the prevention of neuroblastoma relapse. Cancer Lett 2003; 197: 205-9.
12. Munn D H, Cheung N K. Phagocytosis of tumor cells by human monocytes cultured in recombinant macrophage colony-stimulating factor. J Exp Med 1990; 172: 231-7.
13. Jaiswal S, Jamieson C H, Pang W W, Park C Y, Chao M P, Majeti R, Traver D, van Rooijen N, Weissman I L. CD47 is upregulated on circulating hematopoietic stem cells and leukemia cells to avoid phagocytosis. Cell 2009; 138: 271-85.
14. Barclay A N, Van den Berg T K. The interaction between signal regulatory protein alpha (SIRPalpha) and CD47: structure, function, and therapeutic target. Annu Rev Immunol 2014; 32: 25-50.
15. Majeti R, Chao M P, Alizadeh A A, Pang W W, Jaiswal S, Gibbs K D, Jr., van Rooijen N, Weissman I L. CD47 is an adverse prognostic factor and therapeutic antibody target on human acute myeloid leukemia stem cells. Cell 2009; 138: 286-99.
16. Weiskopf K, Ring A M, Ho C C, Volkmer J P, Levin A M, Volkmer A K, Ozkan E, Fernhoff N B, van de Rijn M, Weissman I L, Garcia K C. Engineered SIRPalpha variants as immunotherapeutic adjuvants to anticancer antibodies. Science 2013; 341: 88-91.
17. Chao M P, Alizadeh A A, Tang C, Myklebust J H, Varghese B, Gill S, Jan M, Cha A C, Chan C K, Tan B T, Park C Y, Zhao F, et al. Anti-CD47 antibody synergizes with rituximab to promote phagocytosis and eradicate non-Hodgkin lymphoma. Cell 2010; 142: 699-713.
18. Petrova P S, Viller N N, Wong M, Pang X, Lin G H, Dodge K, Chai V, Chen H, Lee V, House V, Vigo N T, Jin D, et al. TTI-621 (SIRPalphaFc): A CD47-Blocking Innate Immune Checkpoint Inhibitor with Broad Antitumor Activity and Minimal Erythrocyte Binding. Clin Cancer Res 2017; 23: 1068-79.
19. Yanagita T, Murata Y, Tanaka D, Motegi S I, Arai E, Daniwijaya E W, Hazama D, Washio K, Saito Y, Kotani T, Ohnishi H, Oldenborg P A, et al. Anti-SIRPalpha antibodies as a potential new tool for cancer immunotherapy. JCI Insight 2017; 2: e89140.
20. Alvarez-Rueda N, Desselle A, Cochonneau D, Chaumette T, Clemenceau B, Leprieur S, Bougras G, Supiot S, Mussini J M, Barbet J, Saba J, Paris F, et al. A monoclonal antibody to O-acetyl-GD2 ganglioside and not to GD2 shows potent anti-tumor activity without peripheral nervous system cross-reactivity. PLOS One 2011; 6: e25220.
21. Cochonneau D, Terme M, Michaud A, Dorvillius M, Gautier N, Frikeche J, Alvarez-Rueda N, Bougras G, Aubry J, Paris F, Birkle S. Cell cycle arrest and apoptosis induced by O-acetyl-GD2-specific monoclonal antibody 8B6 inhibits tumor growth in vitro and in vivo. Cancer Lett 2013; 333: 194-204.
22. Terme M, Dorvillius M, Cochonneau D, Chaumette T, Xiao W, Diccianni M B, Barbet J, Yu A L, Paris F, Sorkin L S, Birkle S. Chimeric antibody c.8B6 to O-acetyl-GD2 mediates the same efficient anti-neuroblastoma effects as therapeutic ch14.18 antibody to GD2 without antibody induced allodynia. PLOS One 2014; 9: e87210.
23. Nair A B, Jacob S. A simple practice guide for dose conversion between animals and human. J Basic Clin Pharm 2016; 7: 27-31.
24. Fleurence Julien et al. 2016. Targeting and killing glioblastoma with monoclonal antibody to O-acetyl GD2 ganglioside. Oncotarget.
25. Chuang W, Lagenaur C F. Central nervous system antigen P84 can serve as a substrate for neurite outgrowth. Dev Biol 1990; 137: 219-32.
26. Trouplin V, Boucherit N, Gorvel L, Conti F, Mottola G, Ghigo E. Bone marrow-derived macrophage production. J Vis Exp 2013: e50966.
27. Shi Y, Fan X, Deng H, Brezski R J, Rycyzyn M, Jordan R E, Strohl W R, Zou Q, Zhang N, An Z. Trastuzumab triggers phagocytic killing of high HER2 cancer cells in vitro and in vivo by interaction with Fcgamma receptors on macrophages. J Immunol 2015; 194: 4379-86.

1-15. (canceled)

16. A method for treating a cancer comprising administering to a subject in need thereof a pharmaceutical composition comprising an anti-O-acetylated disialoganglioside (OAcGD2) compound and an anti-SIRP-alpha or anti-CD47 compound.

17. The method according to claim 16, wherein said pharmaceutical composition further comprises at least one anti-cancer agent.

18. A method for treating a cancer comprising administering to a subject in need thereof a therapeutically effective amount of an anti-O-acetylated disialoganglioside (OAcGD2) compound and an anti-SIRP-alpha or anti-CD47 compound.

19. The method according to claim 18, wherein the cancer is a neuroblastoma, a glioblastoma, a small-cell lung carcinoma or a breast cancer.

20. The method according to claim 18, wherein the cancer is a neuroblastoma.

21. The method according to claim 18, wherein the anti-OAcGD2 compound is an antibody comprising the following complementary-determining regions (CDRs):
CDR1:
(SEQ ID NO: 1)
EFTFTDYY;
CDR2:
(SEQ ID NO: 2)
IRNRANGYTT;
CDR3:
(SEQ ID NO: 3)
ARVSNWAFDY;
CDR4:
(SEQ ID NO: 4)
QSLLKNNGNTFL;
CDR5:
(SEQ ID NO: 5)
KVS;
and
CDR6:
(SEQ ID NO: 6)
SQSTHIPYT.

22. The method according to claim 18, wherein the anti-OAcGD2 compound is a humanized antibody.

23. The method according to claim 18, wherein the anti-OAcGD2 compound is an antibody comprising:
a) a light chain variable region (VL) having the amino acid sequence SEQ ID NO: 7, or a sequence having a percentage of identity of at least 85% with SEQ ID NO: 7; and
b) a heavy chain variable region (VH) having the amino acid sequence SEQ ID NO: 8, or a sequence having a percentage of identity of at least 85% with SEQ ID NO: 8.

24. The method according to claim 18, wherein the anti-OAcGD2 compound is an antibody having a sequence comprising:
a heavy chain variable region (VH) sequence selected from the group consisting of SEQ ID NO: 9 (“VH49A”), SEQ ID NO: 10 (“VH72A”), SEQ ID NO: 11 (“VH49BHS”), SEQ ID NO: 12 (“VH72BHNPS”), SEQ ID NO: 16 (“VH49BHSs”), SEQ ID NO: 17 (“VH72BHNPSs”), or a variant thereof; and/or
a light chain variable region (VL) sequence selected from the group consisting of SEQ ID NO: 13 (“VL30A”), SEQ ID NO: 14 (“VL28A”), SEQ ID NO: 15 (“VL28Bs01/A2”), SEQ ID NO: 18 (“VL30As”), SEQ ID NO: 19 (“VL28B01/A2”), or a variant thereof.

25. The method according to claim 18, wherein the anti-O-acetylated disialoganglioside (OAcGD2) compound and the anti-SIRP-alpha or anti-CD47 compound are a multi-specific antibody having at least two antigen binding sites directed to OAcGD2 and to SIRP-alpha or CD47.

26. The method according to claim 25, wherein the multi-specific antibody is a bi-specific antibody directed to OAcGD2 and to SIRP-alpha or CD47 or a derivative thereof.

27. The method according to claim 25, wherein the multi-specific antibody is a trivalent or tetravalent antibody comprising at least two antigen binding sites directed to OAcGD2 and at least one antigen binding site directed to SIRP-alpha or CD47.

28. The method according to claim 18, further comprising administering to the subject in need thereof a therapeutically effective amount of a retinoic acid.

29. The method according to claim 17, wherein the at least one anti-cancer agent is interleukin-2 (IL-2) and/or an anti-disialoganglioside (anti-GD2) antibody.

30. The method according to claim 18, wherein the anti-O-acetylated disialoganglioside (OAcGD2) compound and the anti-SIRP-alpha or anti-CD47 compound are administered simultaneously, separately, or sequentially.


SEQUENCE LISTING (38 sequences)

SEQ ID NO: 1 - protein, 8 aa, Artificial Sequence
  8B6 VH-CDR1
EFTFTDYY

SEQ ID NO: 2 - protein, 10 aa, Artificial Sequence
  8B6 VH-CDR2
IRNRANGYTT

SEQ ID NO: 3 - protein, 10 aa, Artificial Sequence
  8B6 VH-CDR3
ARVSNWAFDY

SEQ ID NO: 4 - protein, 12 aa, Artificial Sequence
  8B6 VL-CDR1
QSLLKNNGNTFL

SEQ ID NO: 5 - protein, 3 aa, Artificial Sequence
  8B6 VL-CDR2
KVS

SEQ ID NO: 6 - protein, 9 aa, Artificial Sequence
  8B6 VL-CDR3
SQSTHIPYT

SEQ ID NO: 7 - protein, 112 aa, Artificial Sequence
  SITE 1..1: X=D or E
  Humanized anti-OAcGD2 VL consensus sequence
  SITE 2..2: X=V or I
  SITE 9..9: X=L or A
  SITE 10..10: X=S or T
  SITE 12..12: X=P or S
  SITE 13..13: X=V or L or A
  SITE 14..14: X=S or T
  SITE 15..15: X=L or P or V
  SITE 17..17: X=D or Q or E
  SITE 18..18: X=Q or P or R
  SITE 19..19: X=A or V
  SITE 20..20: X=S or T
  SITE 21..21: X=I or L
  SITE 22..22: X=S or T
  SITE 25..25: X=S or A
  SITE 29..29: X=L or V
  SITE 30..30: X=L or V
  SITE 32..32: X=N or S
  SITE 33..33: X=N or Q
  SITE 34..34: X=G or A or S
  SITE 35..35: X=N or Y or S
  SITE 36..36: X=T or N or S
  SITE 37..37: X=F or Y
  SITE 39..39: X=H or N or S or A or Y or G
  SITE 41..41: X=Y or F
  SITE 42..42: X=L or Q
  SITE 44..44: X=K or R
  SITE 47..47: X=Q or K
  SITE 48..48: X=S or A or V
  SITE 50..50: X=K or Q or R
  SITE 51..51: X=L or R or V
  SITE 55..55: X=K or G or L
  SITE 56..56: X=V or A or G
  SITE 58..58: X=N or T
  SITE 60..60: X=L or D or A
  SITE 61..61: X=S or T
  SITE 63..63: X=V or I
  SITE 65..65: X=D or A or S
  SITE 75..75: X=Y or D
  SITE 79..79: X=K or T
  SITE 81..81: X=S or N
  SITE 82..82: X=R or S
  SITE 83..83: X=V or L
  SITE 84..84: X=E or Q
  SITE 85..85: X=A or P
  SITE 88..88: X=L or V or F
  SITE 89..89: X=G or A
  SITE 90..90: X=V or T
  SITE 92..92: X=F or Y
  SITE 94..94: X=S or M or Q
  SITE 96..96: X=S or A
  SITE 97..97: X=T or Y
  SITE 98..98: X=H or Q or N
  SITE 99..99: X=I or T or Q
  SITE 100..100: X=P or S
  SITE 105..105: X=G or Q
XXVMTQSPXXLXXXXGXXXXXXCRXSQSXXKXXXXXXLXWXXQXPGXXPXXLIYXXSXRXXGXPXRFSGSGSGTXFTLXIXXXXXEDXXXYXCXQXXXXXYTFGXGTKVEIK

SEQ ID NO: 8 - protein, 119 aa, Artificial Sequence
  SITE 1..1: X=E or Q
  Humanized anti-OAcGD2 VH consensus sequence
  SITE 5..5: X=V or L
  SITE 13..13: X=Q or K
  SITE 16..16: X=G or R
  SITE 23..23: X=A or T
  SITE 24..24: X=T or A
  SITE 26..26: X=E or G
  SITE 30..30: X=T or S or G
  SITE 32..32: X=Y or H
  SITE 35..35: X=T or H or N or S
  SITE 37..37: X=V or I
  SITE 48..48: X= L or V
  SITE 49..49: X= G or S
  SITE 50..50: X= F or Y
  SITE 51..51: X= I or T
  SITE 54..54: X= R or K or S
  SITE 55..55: X= A or S
  SITE 56..56: X=N or S
  SITE 57..57: X= G or A or S
  SITE 58..58: X= Y or G
  SITE 59..59: X= T or I
  SITE 60..60: X= T or I
  SITE 61..61: X= E or Y
  SITE 63..63: X= N or A
  SITE 64..64: X= P or A or D
  SITE 76..76: X= N or G
  SITE 77..77: X= S or A
  SITE 79..79: X= S or N
  SITE 80..80: X= I or S or T
  SITE 81..81: X= L or T or A
  SITE 89..89: X= R or K or Q
  SITE 90..90: X= T or A
  SITE 95..95: X= V or I or L
  SITE 99..99: X= A or T
  SITE 105..105: X=A or Y
  SITE 114..114: X= T or L
  SITE 115..115: X= L or V
XVQLXESGGGLVXPGXSLRLSCXXSXFTFXDXYMXWXRQAPGKGLEWXXXXRNXXXXXXXXYXXSVKGRFTISRDXXKXXXYLQMNSLXXEDTAXYYCXRVSNWXFDYWGQGTXXTVSS

SEQ ID NO: 9 - protein, 119 aa, Artificial Sequence
  Anti-OAcGD2 VH49A
EVQLVESGGGLVQPGRSLRLSCTTSEFTFTDYYMTWVRQAPGKGLEWLGFIRNRANGYTTEYNPSVKGRFTISRDNSKSILYLQMNSLKTEDTAVYYCARVSNWAFDYWGQGTLVTVSS

SEQ ID NO: 10 - protein, 119 aa, Artificial Sequence
  Anti-OAcGD2 VH72A
EVQLVESGGGLVQPGGSLRLSCATSEFTFTDYYMTWVRQAPGKGLEWLGFIRNRANGYTTEYNPSVKGRFTISRDNSKNSLYLQMNSLKTEDTAVYYCARVSNWAFDYWGQGTLVTVSS

SEQ ID NO: 11 - protein, 119 aa, Artificial Sequence
  Anti-OAcGD2 VH49BHS
EVQLVESGGGLVQPGRSLRLSCTTSEFTFTDYYMTWVRQAPGKGLEWLGFIRNKANGYTTEYNPSVKGRFTISRDNSKSILYLQMNSLKTEDTAVYYCARVSNWAFDYWGQGTLVTVSS

SEQ ID NO: 12 - protein, 119 aa, Artificial Sequence
  Anti-OAcGD2 VH72BHNPS
EVQLVESGGGLVQPGGSLRLSCATSEFTFSDYYMTWVRQAPGKGLEWLGFIRNKANGYTTEYNPSVKGRFTISRDNSKNSLYLQMNSLKTEDTAVYYCARVSNWAFDYWGQGTLVTVSS

SEQ ID NO: 13 - protein, 112 aa, Artificial Sequence
  Anti-OAcGD2 VL30A
DVVMTQSPLSLPVTLGQPASISCRSSQSLLKNNGNTFLHWYQQRPGQSPRLLIYKVSNRLSGVPDRFSGSGSGTDFTLKISRVEAEDVGVYFCSQSTHIPYTFGGGTKVEIK

SEQ ID NO: 14 - protein, 112 aa, Artificial Sequence
  Anti-OAcGD2 VL28A
DVVMTQSPLSLPVTPGEPASISCRSSQSLLKNNGNTFLHWYLQKPGQSPQLLIYKVSNRLSGVPDRFSGSGSGTDFTLKISRVEAEDVGVYFCSQSTHIPYTFGQGTKVEIK

SEQ ID NO: 15 - protein, 112 aa, Artificial Sequence
  Anti-OAcGD2 VL28Bs01/A2
DVVMTQSPLSLPVTPGEPASISCRSSQSLLKSNANTFLHWYLQKPGQSPQLLIYKVSNRLSGVPDRFSGSGSGTDFTLKISRVEAEDVGVYYCMQSTHIPYTFGQGTKVEIK

SEQ ID NO: 16 - protein, 119 aa, Artificial Sequence
  Anti-OAcGD2 VH49BHSs
EVQLVESGGGLVQPGRSLRLSCTTSEFTFTDYYMTWVRQAPGKGLEWLGFIRNKASGYTTEYNPSVKGRFTISRDNSKSILYLQMNSLKTEDTAVYYCARVSNWAFDYWGQGTLVTVSS

SEQ ID NO: 17 - protein, 119 aa, Artificial Sequence
  Anti-OAcGD2 VH72BHNPSs
EVQLVESGGGLVQPGGSLRLSCATSEFTFSDYYMTWVRQAPGKGLEWLGFIRNKANSYTTEYNPSVKGRFTISRDNSKNSLYLQMNSLKTEDTAVYYCARVSNWAFDYWGQGTLVTVSS

SEQ ID NO: 18 - protein, 112 aa, Artificial Sequence
  Anti-OAcGD2 VL30As
DVVMTQSPLSLPVTLGQPASISCRSSQSLLKNQGNTFLHWYQQRPGQSPRLLIYKVSNRLSGVPDRFSGSGSGTDFTLKISRVEAEDVGVYFCSQSTHIPYTFGGGTKVEIK

SEQ ID NO: 19 - protein, 112 aa, Artificial Sequence
  Anti-OAcGD2 VL28B01/A2
DVVMTQSPLSLPVTPGEPASISCRSSQSLLKSNGNTFLHWYLQKPGQSPQLLIYKVSNRLSGVPDRFSGSGSGTDFTLKISRVEAEDVGVYYCMQSTHIPYTFGQGTKVEIK

SEQ ID NO: 20 - protein, 449 aa, Artificial Sequence
  Anti-OAcGD2 HC (anti-OAcGD2 anti-CD47 bispecific)
EVQLVESGGGLVQPGGSLRLSCATSEFTFSDYYMTWVRQAPGKGLEWLGFIRNKANSYTTEYNPSVKGRFTISRDNSKNSLYLQMNSLKTEDTAVYYCARVSNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 21 - protein, 219 aa, Artificial Sequence
  Anti-OAcGD2 LC (anti-OAcGD2 anti-CD47 bispecific)
DVVMTQSPLSLPVTLGQPASISCRSSQSLLKNNGNTFLHWYQQRPGQSPRLLIYKVSNRLSGVPDRFSGSGSGTDFTLKISRVEAEDVGVYFCSQSTHIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 355 aa, Artificial Sequence
  Anti-CD47 VHH (anti-OAcGD2 anti-CD47 bispecific)
QVQLVESGGGLVEPGGSLRLSCAASGIIFKINDMGWYRQAPGKRREWVAASTGGDEAIYRDSVKDRFTISRDAKNSVFLQMNSLKPEDTAVYYCTAVISTDRDGTEWRRYWGQGTQVTVSSGGGGGGSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSRYTQKSLSLSPGK

SEQ ID NO: 23 - protein, 449 aa, Artificial Sequence
  Anti-DOTA HC (anti-DOTA anti-CD47 bispecific)
EVMLVESGGGLVKPGGSLTLSCAASGFTFTAHAMSWVRQTPEKRLEWVATISGGGTYTYFPDSFQGRFTISSDNAKNTLYLQMSSLRSEDTSMYFCTRHGDYRYAFGYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 24 - protein, 216 aa, Artificial Sequence
  Anti-DOTA LC (anti-DOTA anti-CD47 bispecific)
QAVTQESALTTSPGETVTLTCRSSSGAVTTNNYANWVQEKPDHLFTGLIGGTNNRAPGVPARFSGSLIGDKAALTITGAQTEDEAIYFCALWFSDHWVFGGGTELTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 355 aa, Artificial Sequence; identical to SEQ ID NO: 22
  Anti-CD47 VHH (anti-DOTA anti-CD47 bispecific)

SEQ ID NO: 26 - protein, 449 aa, Artificial Sequence; identical to SEQ ID NO: 20
  Anti-OAcGD2 HC (anti-OAcGD2 anti-DOTA bispecific)

SEQ ID NO: 27 - protein, 219 aa, Artificial Sequence; identical to SEQ ID NO: 21
  Anti-OAcGD2 LC (anti-OAcGD2 anti-DOTA bispecific)

SEQ ID NO: 28 - protein, 453 aa, Artificial Sequence
  Anti-DOTA HC (anti-OAcGD2 anti-DOTA bispecific)
EVMLVESGGGLVKPGGSLTLSCAASGFTFTAHAMSWVRQTPEKRLEWVATISGGGTYTYFPDSFQGRFTISSDNAKNTLYLQMSSLRSEDTSMYFCTRHGDYRYAFGYWGQGTLVTVSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSRYTQKSLSLSPGK

SEQ ID NO: 29 - protein, 212 aa, Artificial Sequence
  Anti-DOTA LC (anti-OAcGD2 anti-DOTA bispecific)
QAVTQESALTTSPGETVTLTCRSSSGAVTTNNYANWVQEKPDHLFTGLIGGTNNRAPGVPARFSGSLIGDKAALTITGAQTEDEAIYFCALWFSDHWVFGGGTELTVLGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 30 - protein, 449 aa, Artificial Sequence
  Anti-OAcGD2 HC (monospecific)
EVQLVESGGGLVQPGGSLRLSCATSEFTFSDYYMTWVRQAPGKGLEWLGFIRNKANSYTTEYNPSVKGRFTISRDNSKNSLYLQMNSLKTEDTAVYYCARVSNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 31 - protein, 219 aa, Artificial Sequence; identical to SEQ ID NO: 21
  Anti-OAcGD2 LC (monospecific)

SEQ ID NO: 32 - protein, 449 aa, Artificial Sequence
  Anti-DOTA HC (monospecific)
EVMLVESGGGLVKPGGSLTLSCAASGFTFTAHAMSWVRQTPEKRLEWVATISGGGTYTYFPDSFQGRFTISSDNAKNTLYLQMSSLRSEDTSMYFCTRHGDYRYAFGYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 33 - protein, 216 aa, Artificial Sequence; identical to SEQ ID NO: 24
  Anti-DOTA LC (monospecific)

SEQ ID NO: 34 - protein, 355 aa, Artificial Sequence
  Anti-CD47 HC (monospecific)
QVQLVESGGGLVEPGGSLRLSCAASGIIFKINDMGWYRQAPGKRREWVAASTGGDEAIYRDSVKDRFTISRDAKNSVFLQMNSLKPEDTAVYYCTAVISTDRDGTEWRRYWGQGTQVTVSSGGGGGGSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 35 - protein, 8 aa, Artificial Sequence
  Anti-CD47 VHH-CDR1
GIIFKIND

SEQ ID NO: 36 - protein, 8 aa, Artificial Sequence
  Anti-CD47 VHH-CDR2
ASTGGDEA

SEQ ID NO: 37 - protein, 15 aa, Artificial Sequence
  Anti-CD47 VHH-CDR3
TAVISTDRDGTEWRR

SEQ ID NO: 38 - protein, 121 aa, Artificial Sequence
  Anti-CD47 VHH
QVQLVESGGGLVEPGGSLRLSCAASGIIFKINDMGWYRQAPGKRREWVAASTGGDEAIYRDSVKDRFTISRDAKNSVFLQMNSLKPEDTAVYYCTAVISTDRDGTEWRRYWGQGTQVTVSS